(19) United States
(12) Patent Application Publication
(10) Pub. No.: US 2009/0182033 A1
(43) Pub. Date: Jul. 16, 2009

(54) Cyclic Amine Derivatives and Their Uses

(75) Inventors: Harry Finch, Harlow (GB); Nicholas Charles Ray, Harlow (GB); Monique Bodil Van Niel, Harlow (GB); Phillip Smith, Harlow (GB)

Correspondence Address: FISH & RICHARDSON P.C., P.O BOX 1022, MINNEAPOLIS, MN, 55440-1022, US

(73) Assignee: ARGENTA DISCOVERY LTD., Harlow (GB)

(21) Appl. No.: 12/096,563

(22) PCT Filed: Dec. 14, 2006
(86) PCT No.: PCT/GB2006/004675

§ 371 Date: Dec. 3, 2008

(30) Foreign Application Priority Data
Dec. 16, 2005 (GB) 0525675.5
Sep. 29, 2006 (GB) 0619309.8

(51) Int. Cl. A61K 31/381; C07D 409/06; C07C 211/40; C07C 69/76; C07D 311/84; A61K 31/352; A61K 31/24; A61K 31/135; A61P 1/00; A61P 11/00; A61P 9/00; A61P 11/06; A61P 11/08; A61P 11/02

(52) U.S. Cl. 514/444; 549/59; 564/305; 560/58; 549/393; 514/455; 514/534; 514/646

(57) ABSTRACT

Compounds of formula (I) have muscarinic M3 receptor modulating activity; formula (I) wherein R¹ is C₁-C₆-alkyl or a hydrogen atom; and R² is a hydrogen atom or a group -R⁵ or a group, -Z-Y—R⁵, or a group -Z-NR⁹R¹⁰, or a group -Z-N(R⁹)C(O)R¹¹; and R³ is a lone pair, or C₁-C₆-alkyl; R⁴ is selected from one of the groups of formula (a), (b), (c) or (d); formulae (a), (b), (c), (d), Z is a C₁-C₁₆-alkylene, C₂-C₁₆-alkenylene or C₂-C₁₆-alkynylene group; Y is a bond or oxygen atom; R⁵ is an C₁-C₆-alkyl, aryl, arylalkyl; aryl-fused-cycloalkyl, aryl-fused-heterocycloalkyl, heteroaryl, aryl(C₁-C₈-alkyl)-, heteroaryl(C₁-C₈-alkyl)-, cycloalkyl or heterocycloalkyl group; R⁶ is C₁-C₆-alkyl or a hydrogen atom; R^{7a} and R^{7b} area C₁-C₆-alkyl group or halogen; n and m are independently 0, 1, 2 or 3; R^{8a} and R^{8b} are independently selected from the group consisting of aryl, aryl-fused-heterocycloalkyl, heteroaryl, C₁-C₆-alkyl, cycloalkyl and hydrogen; R^{8c} is —OH, C₁-C₆-alkyl, hydroxy-C₁-C₆-alkyl, or a hydrogen atom; R^{8d} is C₁-C₆-alkyl or a hydrogen atom; R⁹ and R¹⁰ are independently a hydrogen atom, C₁-C₆-alkyl, aryl aryl-fused-heterocycloalkyl, aryl-fused-cycloalkyl, heteroaryl, aryl(C₁-C₆-alkyl)-, or heteroaryl(C₁-C₆-alkyl)-group; or R⁹ and R¹⁰ together with the nitrogen atom to which they are attached form a heterocyclic ring of 4-8 atoms, optionally containing a further nitrogen or oxygen atom; R¹¹ is C₁-C₆-alkyl or a hydrogen atom; Ar¹ is aryl, heteroaryl or cycloalkyl; Ar² are independently aryl, heteroaryl or cycloalkyl; and Q is an oxygen atom, —CH₂—, —CH₂CH₂— or a bond.

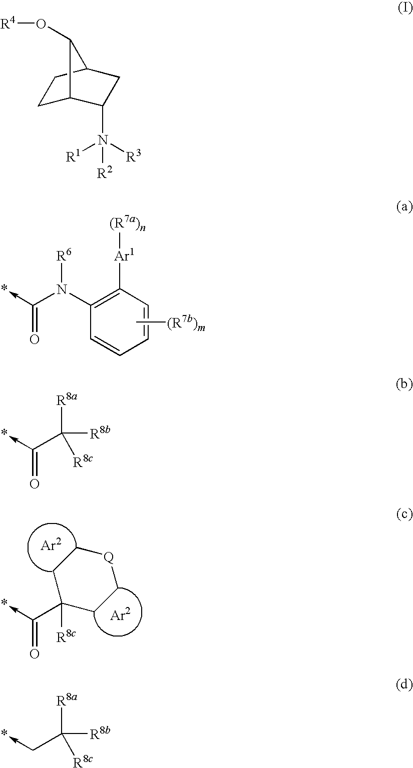

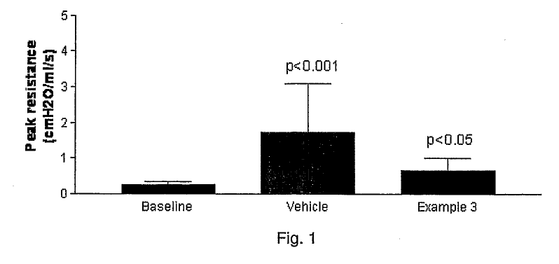

### FIELD OF THE INVENTION

This invention relates to bicyclo[2.2.1]hept-2-ylamine derivatives, pharmaceutical compositions, methods for their preparation and use in the treatment of M3 muscarinic receptor mediated diseases, for example respiratory diseases.

### BACKGROUND TO THE INVENTION

Anti-cholinergic agents prevent the passage of, or effects resulting from the passage of, impulses through the parasympathetic nerves. This is a consequence of the ability of such compounds to inhibit the action of acetylcholine (Ach) by blocking its binding to the muscarinic cholinergic receptors.

There are five subtypes of muscarinic acetylcholine receptors (mAChRs), termed M1-M5, and each is the product of a distinct gene and each displays unique pharmacological properties. mAChRs are widely distributed in vertebrate organs, and these receptors can mediate both inhibitory and excitatory actions. For example, in smooth muscle found in the airways, bladder and gastrointestinal tract, M3 mAChRs mediate contractile responses (reviewed by Caulfield, 1993, Pharmac. Ther., 58, 319-379).

In the lungs, muscarinic receptors M1, M2 and M3 have been demonstrated to be important and are localized to the trachea, the bronchi, submucosal glands and parasympathetic ganglia (reviewed in Fryer and Jacoby, 1998, Am J Resp Crit. Care Med., 158 (5 part 3) S 154-160). M3 receptors on airway smooth muscle mediate contraction and therefore bronchoconstriction. Stimulation of M3 receptors localised to submucosal glands results in mucus secretion.

Increased signalling through muscarinic acetylcholine receptors has been noted in a variety of different pathophysiological states including asthma and COPD. In COPD, vagal tone may either be increased (Gross et al. 1989, Chest; 96:984-987) and/or may provoke a higher degree of obstruction for geometric reasons if applied on top of oedematous or mucus-laden airway walls (Gross et al. 1984, Am Rev Respir Dis; 129:856-870). In addition, inflammatory conditions can lead to a loss of inhibitory M2 receptor activity which results in increased levels of acetylcholine release following vagal nerve stimulation (Fryer et al, 1999, Life Sci., 64, (6-7) 449-455). The resultant increased activation of M3 receptors leads to enhanced airway obstruction. Thus the identification of potent muscarinic receptor antagonists would be useful for the therapeutic treatment of those disease states where enhanced M3 receptor activity is implicated. Indeed, contemporary treatment strategies currently support regular use of M3 antagonist bronchodilators as first-line therapy for COPD patients (Pauwels et al. 2001, Am Rev Respir Crit. Care Med; 163:1256-1276)

Incontinence due to bladder hypercontractility has also been demonstrated to be mediated through increased stimulation of M3 mAChRs. Thus M3 mAChR antagonists may be useful as therapeutics in these mAChR-mediated diseases.

Despite the large body of evidence supporting the use of anti-muscarinic receptor therapy for treatment of airway disease states, relatively few anti-muscarinic compounds are in use in the clinic for pulmonary indications. Thus, there remains a need for novel compounds that are capable of causing blockade at M3 muscarinic receptors, especially those compounds with a long duration of action, enabling a once-daily dosing regimen. Since muscarinic receptors are widely distributed throughout the body, the ability to deliver anticholinergic drugs directly to the respiratory tract is advantageous as it allows lower doses of the drug to be administered. The design and use of topically active drugs with a long duration of action and that are retained on the receptor or in the lung would allow reduction of unwanted side effects that could be seen with systemic administration of the same drugs.

Tiotropium (Spiriva™) is a long-acting muscarinic antagonist currently marketed for the treatment of chronic obstructive pulmonary disease, administered by the inhaled route.

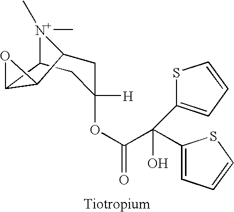

Additionally ipratropium is a muscarinic antagonist marketed for the treatment of COPD.

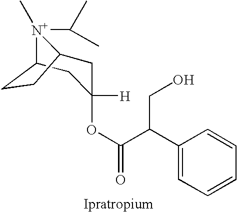

Other muscarinic receptor modulators have been referred to. For example: U.S. Pat. No. 4,353,922 describes muscarinic modulators based upon a [2.2.1]azabicycloheptane ring system. EP418716 and US005610163 describe various [3.2.1]azabicyclooctane ring systems. WO06/017768 describes [3.3.1]azabicyclononane ring systems. [2.2.2]azabicyclooctane systems (quinuclidines) have been previously described, for example in US2005/0209272 and WO06/048225. [3.1.0]azabicyclohexane systems have been described in, for example in WO06/035282. [3.2.1]azabicyclooctane systems have been described in for example WO06/035303.

### SUMMARY OF THE INVENTION

According to the invention, there is provided a compound of formula (I):

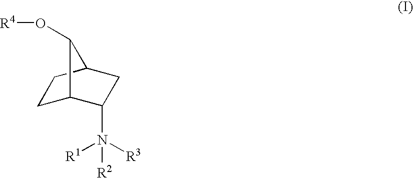

wherein
R¹ is C₁-C₆-alkyl or a hydrogen atom; and R² is a hydrogen atom or a group —R⁵, or a group, -Z-Y—R⁵, or a group -Z-NR⁹R¹⁰, or a group -Z-N(R⁹)C(O)R¹¹; and R³ is a lone pair, or C₁-C₆-alkyl in which case the nitrogen atom to which it is attached is a quaternary nitrogen and carries a positive charge;
R⁴ is selected from one of the groups of formula (a), (b), (c) or (d);

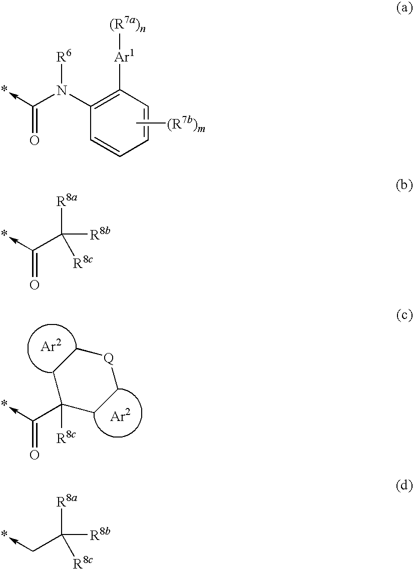

Z is a C₁-C₁₆-alkylene, C₂-C₁₆-alkenylene or C₂-C₁₆-alkynylene group;
Y is a bond or oxygen atom;

R⁵ is an C₁-C₆-alkyl, aryl, arylalkyl; aryl-fused-cycloalkyl, aryl-fused-heterocycloalkyl, heteroaryl, aryl(C₁-C₈-alkyl)-, heteroaryl(C₁-C₈-alkyl)-, cycloalkyl or heterocycloalkyl group;

R⁶ is C₁-C₆-alkyl or a hydrogen atom;
R^{7a} and R^{7b} are a C₁-C₆-alkyl group or halogen;
n and m are independently 0, 1, 2 or 3;
R^{8a} and R^{8b} are independently selected from the group consisting of aryl, aryl-fused-heterocycloalkyl, heteroaryl, C₁-C₆-alkyl, cycloalkyl and hydrogen;
R^{8c} is —OH, C₁-C₆-alkyl, hydroxy-C₁-C₆-alkyl, or a hydrogen atom;
R⁹ and R¹⁰ are independently a hydrogen atom, C₁-C₆-alkyl, aryl, aryl-fused-heterocycloalkyl, aryl-fused-cycloalkyl, heteroaryl, aryl(C₁-C₆-alkyl)-, or heteroaryl(C₁-C₆-alkyl)-group; or R⁹ and R¹⁰ together with the nitrogen atom to which they are attached form a heterocyclic ring of 4-8 atoms, optionally containing a further nitrogen or oxygen atom;
R¹¹ is C₁-C₆-alkyl or a hydrogen atom;
Ar¹ is aryl, heteroaryl or cycloalkyl;
Ar² are independently aryl, heteroaryl or cycloalkyl; and
Q is an oxygen atom, —CH₂—, —CH₂CH₂— or a bond;
or a pharmaceutically acceptable salt, solvate, N-oxide or prodrug thereof.

In one subset of the compounds of the invention:

R¹ is C₁-C₆-alkyl or a hydrogen atom and R² is C₁-C₆-alkyl, a hydrogen atom or a group -Z-Y—R⁵, or a group -Z-NR⁹R¹⁰;
R³ is a lone pair; or C₁-C₆-alkyl, in which case the nitrogen atom to which it is attached is a quaternary nitrogen and carries a positive charge;
R⁴ is selected from one of the groups of formula (a) or (b) or (c):

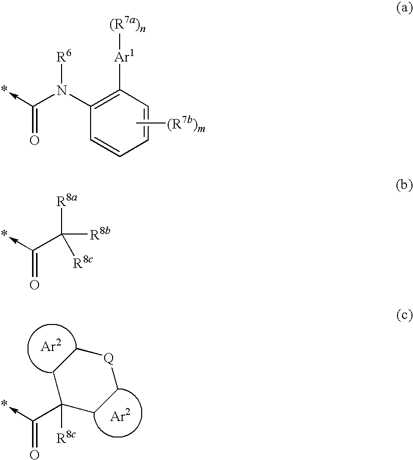

Z is a C₁-C₈-alkylene group;

Y is a bond or oxygen atom;
R⁵ is an aryl or aryl(C₁-C₈-alkyl)-group;
R⁶ is C₁-C₆-alkyl or a hydrogen atom;
R^{7a} and R^{7b} are independently a C₁-C₆-alkyl group or halogen;
n and m are independently 0, 1, 2 or 3;
R^{8a} and R^{8b} are independently selected from the group consisting of aryl, heteroaryl, C₁-C₆-alkyl, cycloalkyl and hydrogen;
R^{8c} is —OH, C₁-C₆-alkyl, hydroxy-C₁-C₆-alkyl, or a hydrogen atom;
R⁹ and R¹⁰ are independently a hydrogen atom, C₁-C₆-alkyl, aryl, heteroaryl, aryl(C₁-C₆-alkyl)-, or heteroaryl(C₁-C₆-alkyl)-group; or R⁹ and R¹⁰ together with the nitrogen atom to which they are attached form a heterocyclic ring of 4-8 atoms, optionally containing a further nitrogen or oxygen atom.

Compounds of the invention exist in either the syn- or anti-forms;

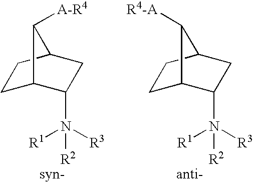

Compounds of the invention also exist with the group —NR¹R²R³ in either the exo or endo orientation;

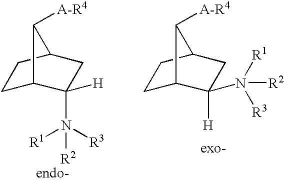

Currently it is preferred that the compounds of the invention be predominantly in the anti-endo configuration.

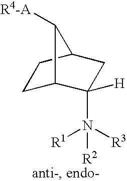

Compounds of the invention can also exist as optical isomers since substituted bicyclic ring systems can lack a plane of symmetry. The absolute configuration of the molecule can be defined using Cahn-Ingold-Prelog rules to assign the R or S designation to each position. To avoid confusion the ring numbering used below is employed.

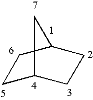

However, compounds of the invention include racemates, single enantiomers and mixtures of the enantiomers in any ratio, since all such forms have muscarinic M3 receptor modulating activity to varying extents.

A preferred class of compounds of the invention consists of quaternary ammonium salts of formula (I) wherein the nitrogen shown in formula (I) is quaternary nitrogen, carrying a positive charge.

Compounds of the invention may be useful in the treatment or prevention of diseases in which activation of muscarinic receptors are implicated, for example the present compounds are useful for treating a variety of indications, including but not limited to respiratory-tract disorders such as chronic obstructive lung disease, chronic bronchitis of all types (including dyspnoea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy, pneumoconiosis (for example aluminosis, anthracosis, asbestosis, chalicosis, ptilosis, siderosis, silicosis, tabacosis and byssinosis);

gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria; motion sickness; and
cardiovascular disorders such as vagally induced sinus bradycardia.

For treatment of respiratory conditions, administration by inhalation will often be preferred, and in such cases administration of compounds (I) which are quaternary ammonium salts will often be preferred. In many cases, the duration of action of quaternary ammonium salts of the invention administered by inhalation is may be more than 12, or more than 24 hours for a typical dose. For treatment of gastrointestinal-tract disorders and cardiovascular disorders, administration by the parenteral route, usually the oral route, may be preferred.

Another aspect of the invention is a pharmaceutical composition comprising a compound of the invention and a pharmaceutically acceptable carrier or excipient.

Another aspect of the invention is the use of a compound of the invention for the manufacture of a medicament for the treatment or prevention of a disease or condition in which muscarinic M3 receptor activity is implicated.

### Terminology

Unless otherwise qualified in the context in which they are used herein, the following terms have the following meanings:
“Acyl” means a —CO-alkyl group in which the alkyl group is as described herein. Exemplary acyl groups include —COCH₃ and —COCH(CH₃)₂.
“Acylamino” means a —NR-acyl group in which R and acyl are as described herein. Exemplary acylamino groups include —NHCOCH₃ and —N(CH₃)COCH₃.
“Alkoxy” and “alkyloxy” means an —O-alkyl group in which alkyl is as described below. Exemplary alkoxy groups include methoxy (—OCH₃) and ethoxy (—OC₂H₅).
“Alkoxycarbonyl” means a —COO-alkyl group in which alkyl is as defined below. Exemplary alkoxycarbonyl groups include methoxycarbonyl and ethoxycarbonyl.
“Alkyl” as a group or part of a group refers to a straight or branched chain saturated hydrocarbon group having from 1 to 12, preferably 1 to 6, carbon atoms, in the chain. Exemplary alkyl groups include methyl, ethyl, 1-propyl and 2-propyl.
“Alkenyl” as a group or part of a group refers to a straight or branched chain hydrocarbon group having from 2 to 12, preferably 2 to 6, carbon atoms and one carbon-carbon double bond in the chain. Exemplary alkenyl groups include ethenyl, 1-propenyl, and 2-propenyl.
“Alkynyl” as a group or part of a group refers to a straight or branched chain hydrocarbon group having from 2 to 12, preferably 2 to 6, carbon atoms and one carbon-carbon triple bond in the chain. Exemplary alkenyl groups include ethynyl, 1-propynyl, and 2-propynyl.
“Alkylamino” means a —NH-alkyl group in which alkyl is as defined above. Exemplary alkylamino groups include methylamino and ethylamino.
“Alkylene means an -alkyl-group in which alkyl is as defined previously. Exemplary alkylene groups include —CH₂—, —(CH₂)₂— and —C(CH₃)HCH₂—.
“Alkenylene” means an -alkenyl-group in which alkenyl is as defined previously. Exemplary alkenylene groups include —CH═CH—, —CH═CHCH₂—, and —CH₂CH═CH—.
“Alkynylene” means an -alkynyl-group in which alkynyl is as defined previously. Exemplary alkenylene groups include —CC—, —CCCH₂—, and —CH₂CC—.
“Alkylsulfinyl” means a —SO-alkyl group in which alkyl is as defined above. Exemplary alkylsulfinyl groups include methylsulfinyl and ethylsulfinyl.
“Alkylsulfonyl” means a —SO₂-alkyl group in which alkyl is as defined above. Exemplary alkylsulfonyl groups include methylsulfonyl and ethylsulfonyl.
“Alkylthio” means a —S-alkyl group in which alkyl is as defined above. Exemplary alkylthio groups include methylthio and ethylthio.
“Aminoacyl” means a —CO—NRR group in which R is as herein described. Exemplary aminoacyl groups include —CONH₂ and —CONHCH₃.
“Aminoalkyl” means an alkyl-NH₂ group in which alkyl is as previously described. Exemplary aminoalkyl groups include —CH₂NH₂.
“Aminosulfonyl” means a —SO₂—NRR group in which R is as herein described. Exemplary aminosulfonyl groups include —SO₂NH₂ and —SO₂NHCH₃.
“Aryl” as a group or part of a group denotes an optionally substituted monocyclic or multicyclic aromatic carbocyclic moiety of from 6 to 14 carbon atoms, preferably from 6 to 10 carbon atoms, such as phenyl or naphthyl. The aryl group may be substituted by one or more substituent groups.
“Arylalkyl” means an aryl-alkyl-group in which the aryl and alkyl moieties are as previously described. Preferred arylalkyl groups contain a C₁₋₄ alkyl moiety. Exemplary arylalkyl groups include benzyl, phenethyl and naphthlenemethyl. The aryl part thereof may be substituted by one or more substituent groups.
“Arylalkyloxy” means an aryl-alkyloxy-group in which the aryl and alkyloxy moieties are as previously described. Preferred arylalkyloxy groups contain a C₁₋₄ alkyl moiety. Exemplary arylalkyl groups include benzyloxy. The aryl part thereof may be substituted by one or more substituent groups.
“Aryl-fused-cycloalkyl” means a monocyclic aryl ring, such as phenyl, fused to a cycloalkyl group, in which the aryl and cycloalkyl are as described herein. Exemplary aryl-fused-cycloalkyl groups include tetrahydronaphthyl and indanyl. The aryl and cycloalkyl rings may each be substituted by one or more substituent groups. The aryl-fused-cycloalkyl group may be attached to the remainder of the compound by any available carbon atom.
“Aryl-fused-heterocycloalkyl” means a monocyclic aryl ring, such as phenyl, fused to a heterocycloalkyl group, in which the aryl and heterocycloalkyl are as described herein. Exemplary aryl-fused-heterocycloalkyl groups include tetrahydroquinolinyl, indolinyl, benzodioxinyl, benzodioxolyl, dihydrobenzofuranyl and isoindolonyl. The aryl and heterocycloalkyl rings may each be substituted by one or more substituent groups. The aryl-fused-heterocycloalkyl group may be attached to the remainder of the compound by any available carbon or nitrogen atom.
“Aryloxy” means an —O-aryl group in which aryl is described above. Exemplary aryloxy groups include phenoxy. The aryl part thereof may be substituted by one or more substituent groups.
“Cyclic amine” is a special case of “Heterocycloalkyl” or “heterocyclic” and means an optionally substituted 3 to 8 membered monocyclic cycloalkyl ring system where one of the ring carbon atoms is replaced by nitrogen, and which may optionally contain an additional heteroatom selected from O, S or NR (where R is as described herein). Exemplary cyclic amines include pyrrolidine, piperidine, morpholine, piperazine and N-methylpiperazine. The cyclic amine group may be substituted by one or more substituent groups.
“Cycloalkyl” means an optionally substituted saturated monocyclic or bicyclic ring system of from 3 to 12 carbon atoms, preferably from 3 to 8 carbon atoms, and more preferably from 3 to 6 carbon atoms. Exemplary monocyclic cycloalkyl rings include cyclopropyl, cyclopentyl, cyclohexyl and cycloheptyl. The cycloalkyl group may be substituted by one or more substituent groups.
“Cycloalkylalkyl” means a cycloalkyl-alkyl-group in which the cycloalkyl and alkyl moieties are as previously described. Exemplary monocyclic cycloalkylalkyl groups include cyclopropylmethyl, cyclopentylmethyl, cyclohexylmethyl and cycloheptylmethyl. The cycloalkyl part thereof may be substituted by one or more substituent groups.
“Dialkylamino” means a —N(alkyl)₂ group in which alkyl is as defined above. Exemplary dialkylamino groups include dimethylamino and diethylamino.
“Halo” or “halogen” means fluoro, chloro, bromo, or iodo. Preferred are fluoro or chloro.
“Haloalkoxy” means an —O-alkyl group in which the alkyl is substituted by one or more halogen atoms. Exemplary haloalkyl groups include trifluoromethoxy and difluoromethoxy.
“Haloalkyl” means an alkyl group which is substituted by one or more halo atoms. Exemplary haloalkyl groups include trifluoromethyl.
“Heteroaryl” as a group or part of a group denotes an optionally substituted aromatic monocyclic or multicyclic organic moiety of from 5 to 14 ring atoms, preferably from 5 to 10 ring atoms, in which one or more of the ring atoms is/are element(s) other than carbon, for example nitrogen, oxygen or sulfur. Examples of such groups include benzimidazolyl, benzoxazolyl, benzothiazolyl, benzofuranyl, benzothienyl, furyl, imidazolyl, indolyl, indolizinyl, isoxazolyl, isoquinolinyl, isothiazolyl, oxazolyl, oxadiazolyl, pyrazinyl, pyridazinyl, pyrazolyl, pyridyl, pyrimidinyl, pyrrolyl, quinazolinyl, quinolinyl, tetrazolyl, 1,3,4-thiadiazolyl, thiazolyl, thienyl and triazolyl groups. The heteroaryl group may be may be substituted by one or more substituent groups. The heteroaryl group may be attached to the remainder of the compound of the invention by any available carbon or nitrogen atom.
“Heteroarylalkyl” means a heteroaryl-alkyl-group in which the heteroaryl and alkyl moieties are as previously described. Preferred heteroarylalkyl groups contain a lower alkyl moiety. Exemplary heteroarylalkyl groups include pyridylmethyl. The heteroaryl part thereof may be substituted by one or more substituent groups.
“Heteroarylalkyloxy” means a heteroaryl-alkyloxy-group in which the heteroaryl and alkyloxy moieties are as previously described. Preferred heteroarylalkyloxy groups contain a lower alkyl moiety. Exemplary heteroarylalkyloxy groups include pyridylmethyloxy. The hetroaryl part thereof may be substituted by one or more substituent groups.
“Heteroaryloxy” means a heteroaryloxy-group in which the heteroaryl is as previously described. Exemplary heteroaryloxy groups include pyridyloxy. The heteroaryl part thereof may be substituted by one or more substituent groups.
“Heteroaryl-fused-cycloalkyl” means a monocyclic heteroaryl group, such as pyridyl or furanyl, fused to a cycloalkyl group, in which heteroaryl and cycloalkyl are as previously described. Exemplary heteroaryl-fused-cycloalkyl groups include tetrahydroquinolinyl and tetrahydrobenzofuranyl. The heteroaryl and cycloalkyl rings may each be substituted by one or more substituent groups. The heteroaryl-fused-cycloalkyl group may be attached to the remainder of the compound by any available carbon or nitrogen atom.
“Heteroaryl-fused-heterocycloalkyl” means a monocyclic heteroaryl group, such as pyridyl or furanyl, fused to a heterocycloalkyl group, in which heteroaryl and heterocycloalkyl are as previously described. Exemplary heteroaryl-fused-heterocycloalkyl groups include dihydrodioxinopyridinyl, dihydropyrrolopyridinyl, dihydrofuranopyridinyl and dioxolopyridinyl. The heteroaryl and heterocycloalkyl rings may each be substituted by one or more substituents groups. The heteroaryl-fused-heterocycloalkyl group may be attached to the remainder of the compound by any available carbon or nitrogen atom.
“Heterocycloalkyl” or “heterocyclic” means: (i) an optionally substituted cycloalkyl group of from 4 to 8 ring members which contains one or more heteroatoms selected from O, S or NR; (ii) a cycloalkyl group of from 4 to 8 ring members which contains CONR and CONRCO (examples of such groups include succinimidyl and 2-oxopyrrolidinyl). The heterocycloalkyl group may be substituted by one or more substituents groups. The heterocycloalkyl group may be attached to the remainder of the compound by any available carbon or nitrogen atom.
“Heterocycloalkylalkyl” or “heterocyclicalkyl” means a heterocycloalkyl-alkyl-group in which the heterocycloalkyl and alkyl moieties are as previously described.
“Lower alkyl” as a group means unless otherwise specified, an aliphatic hydrocarbon group which may be straight or branched having 1 to 4 carbon atoms in the chain, i.e. methyl, ethyl, propyl (propyl or isopropyl) or butyl (butyl, isobutyl or tert-butyl).
“Sulfonyl” means a —SO₂-alkyl group in which alkyl is as described herein. Exemplary sulfonyl groups include methanesulfonyl.
“Sulfonylamino” means a —NR-sulfonyl group in which R and sulfonyl are as described herein. Exemplary sulfonylamino groups include —NHSO₂CH₃. R means alkyl, aryl, or heteroaryl as described herein.
“Pharmaceutically acceptable salt” means a physiologically or toxicologically tolerable salt and includes, when appropriate, pharmaceutically acceptable base addition salts, pharmaceutically acceptable acid addition salts, and pharmaceutically acceptable quaternary ammonium salts. For example (i) where a compound of the invention contains one or more acidic groups, for example carboxy groups, pharmaceutically acceptable base addition salts that may be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that may be formed include hydrochlorides, hydrobromides, sulfates, phosphates, acetates, citrates, lactates, tartrates, mesylates, maleates, fumarates, succinates and the like; (iii) where a compound contains a quaternary ammonium group acceptable counter-ions may be, for example, chlorides, bromides, sulfates, methanesulfonates, benzenesulfonates (besylates), toluenesulfonates (tosylates), napthalene-bissulfonates (napadisylates), phosphates, acetates, citrates, lactates, tartrates, mesylates, maleates, fumarates, succinates and the like. Additional salt forms are detailed in the “Handbook of Pharmaceutical Salts. Properties, selection and use”, P. Heinrich Stahl & Camille G. Wermuth, Wiley-VCH, 2002.
It will be understood that, as used herein, references to the compounds of the invention are meant to also include the pharmaceutically acceptable salts.
“Prodrug” refers to a compound which is convertible in vivo by metabolic means (e.g. by hydrolysis, reduction or oxidation) to a compound of the invention. For example an ester prodrug of a compound of the invention containing a hydroxy group may be convertible by hydrolysis in vivo to the parent molecule. Suitable esters of compounds of the invention containing a hydroxy group, are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and quinates. As another example an ester prodrug of a compound of the invention containing a carboxy group may be convertible by hydrolysis in vivo to the parent molecule. Examples of ester prodrugs are those described by F. J. Leinweber, Drug Metab. Res., 1987, 18, 379.
It will be understood that, as used in herein, references to the compounds of the invention are meant to also include the prodrug forms.
“Saturated” pertains to compounds and/or groups which do not have any carbon-carbon double bonds or carbon-carbon triple bonds.
“Optionally substituted” means optionally substituted with up to four substituents. Optional substituent groups include acyl (e.g. —COCH₃), alkoxy (e.g., —OCH₃), alkoxycarbonyl (e.g. —COOCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoacyl (e.g. —CON(CH₃)₂), aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, —NO₂, aryl (optionally substituted with alkoxy, haloalkoxy, halogen, alkyl or haloalkyl), heteroaryl (optionally substituted with alkoxy, haloalkoxy, halogen, alkyl or haloalkyl), heterocycloalkyl, aminoacyl (e.g. —CONH₂, —CONHCH₃), aminosulfonyl (e.g. —SO₂NH₂, —SO₂NHCH₃), acylamino (e.g. —NHCOCH₃), sulfonylamino (e.g. —NHSO₂CH₃), heteroarylalkyl, cyclic amine (e.g. morpholine), aryloxy, heteroaryloxy, arylalkyloxy (e.g. benzyloxy) and heteroarylalkyloxy.
Alkylene, alkenylene or alkynylene radicals may be optionally substituted. Optional substituent groups in the foregoing radicals include alkoxy (e.g., —OCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, and —NO₂.
Compounds of the invention may exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application of adaptation of known methods (e.g. asymmetric synthesis).

### The Groups R¹, R² and R³

R¹ is C₁-C₆-alkyl or a hydrogen atom; and R² is a hydrogen atom or a group —R⁵, or a group, -Z-Y—R⁵, or a group -Z-NR⁹R¹⁰; or a group -Z-N(R⁹)C(O)R¹¹ and R³ is a lone pair, or C₁-C₆-alkyl in which case the nitrogen atom to which it is attached is quaternary nitrogen and carries a positive charge;

Where a group -R⁵, or a group -Z-Y—R⁵, or a group -Z-NR⁹R¹⁰, or a group -Z-N(R⁹)C(O)R¹¹ is present in R²:
Z may be, for example —(CH₂)₁₋₁₆— the latter being optionally substituted on up to three carbons in the chain by methyl;
Y is a bond or —O—;
R⁵ may be, for example,
   Optionally substituted aryl such as phenyl or naphthyl, or aryl-fused-heterocycloalkyl such as 3,4-methylenedioxyphenyl, 3,4-ethylenedioxyphenyl, or dihydrobenzofuranyl;
   Optionally substituted heteroaryl such as pyridyl, pyrrolyl, pyrimidinyl, oxazolyl, isoxazolyl, benzisoxazolyl, benzoxazolyl, thiazolyl, benzothiazolyl, quinolyl, thienyl, benzothienyl, furyl, benzofuryl, imidazolyl, benzimidazolyl, isothiazolyl, benzisothiazolyl, pyrazolyl, isothiazolyl, triazolyl, benzotriazolyl, thiadiazolyl, oxadiazolyl, pyridazinyl, pyridazinyl, triazinyl, indolyl and indazolyl;
   Optionally substituted aryl(C₁-C₆-alkyl)—such as those wherein the aryl part is any of the foregoing specifically mentioned aryl groups and the —(C₁-C₆-alkyl)-part is —CH₂— or —CH₂CH₂—;
   Optionally substituted aryl-fused-cycloalkyl such as indanyl or 1,2,3,4-tetrahydronaphthalenyl;
   Optionally substituted heteroaryl(C₁-C₈-alkyl)—such as those wherein the heteroaryl part is any of the foregoing specifically mentioned heteroaryl groups and the —(C₁-C₆-alkyl)-part is —CH₂— or —CH₂CH₂—;
   Optionally substituted cycloalkyl such as cyclopropyl, cyclobutyl, cyclopentyl, cyclohexyl, cycloheptyl or cyclooctyl; or
   Optionally substituted heterocycloalkyl(C₁-C₈-alkyl)-, such as those wherein the heterocycloalkyl part is azetidinyl, piperidinyl, piperazinyl, N-substituted piperazinyl such as methylpiperazinyl, or pyrrolidinyl and the —(C₁-C₆-alkyl)-part is —CH₂— or —CH₂CH₂—;
R⁹ and R¹⁰ may be independently selected from hydrogen; C₁-C₆-alkyl such as methyl, ethyl or n- or isopropyl; or any of those optionally substituted aryl, aryl-fused-heterocycloalkyl, aryl-fused-cycloalkyl, heteroaryl or aryl(C₁-C₈-alkyl)-groups specifically mentioned in the discussion of R⁵ above; or
R⁹ and R¹⁰ together with the nitrogen atom to which they are attached may form a heterocyclic ring of 4-8 ring atoms, preferably 4-6 ring atoms optionally containing a further nitrogen or oxygen atom, such as azetidinyl, piperidinyl, piperazinyl, N-substituted piperazinyl such as methylpiperazinyl, pyrrolidinyl, morpholinyl, and thiomorpholinyl;
R¹¹ may be, for example methyl, ethyl or n- or iso-propyl.

Currently preferred are compounds of the invention wherein, in the group —NR¹, R²R³, R¹ is methyl or ethyl, R² is a group -Z-Y—R⁵ as discussed above, especially wherein R⁵ is a cyclic lipophilic group such as phenyl, Y is a bond or —O—, and -Z- is a straight or branched alkylene radical linking the nitrogen and —YR⁵ by a chain of up to 12, for example up to 9, carbon atoms, and R³ is methyl, so that the nitrogen is quaternised and carries a positive charge.

### The Group R⁴

R⁴ is selected from one of the groups of formula (a), (b), (c) or (d);

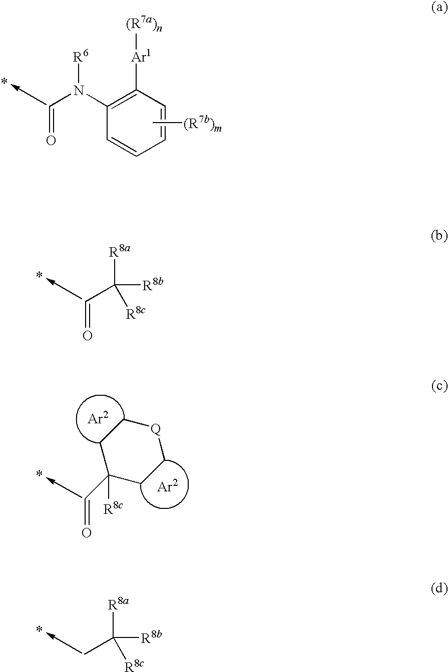

In the group (a), R⁶ may be C₁-C₆-alkyl such as methyl or ethyl or a hydrogen atom; Ar¹ may be an aryl group such as phenyl, a heteroaryl group such as thienyl, especially 2-thienyl, or a cycloalkyl group such as cyclohexyl, cyclopentyl, cyclopropyl, or cyclobutyl; ring substituents R^{7a} and R^{7b} may be independently a C₁-C₆-alkyl group, such as methyl, ethyl, n- or isopropyl, n-, sec- or tertbutyl, or halogen such as fluoro, chloro or bromo; and m and n may be independently 0, 1, 2 or 3.

In the groups (b) and (d), R^{8a} and R^{8b} may be independently selected from any of those aryl, aryl-fused-heterocycloalkyl, aryl-fused-cycloalkyl, heteroaryl, C₁-C₆-alkyl, or cycloalkyl groups specifically mentioned in the discussion of R⁵ above. Additionally, R^{8b} may also be a hydrogen atom. R^{8c} may be —OH, a hydrogen atom, C₁-C₆-alkyl such as methyl or ethyl, or hydroxy-C₁-C₆-alkyl such as hydroxymethyl. Presently preferred is the case where R^{8c} is —OH. Preferred combinations of R^{8a} and R^{8b}, especially when R^{8a} is —OH, include those wherein (i) each of R^{8a} and R^{8b} is optionally substituted monocyclic heteroaryl of 5 or 6 ring atoms such as pyridyl, oxazolyl, thiazolyl, furyl and especially thienyl such a 2-thienyl; (ii) R^{8a} and R^{8b} are both optionally substituted phenyl; (iii) one of R^{8a} and R^{8b} is optionally substituted phenyl and the other is cycloalkyl such as cyclopropyl, cyclobutyl, cycloheptyl, cyclooctyl or especially cyclopentyl or cyclohexyl; and (iv) one of R^{8a} and R^{8b} is optionally substituted monocyclic heteroaryl of 5 or 6 ring atoms such as pyridyl, thienyl, oxazolyl, thiazolyl, or furyl; and the other is cycloalkyl such as cyclopropyl, cyclobutyl, cyclopentyl, cyclohexyl, cycloheptyl or cyclooctyl.

In the group (c), R^{8c} may be —OH, a hydrogen atom, C₁-C₆-alkyl such as methyl or ethyl, or hydroxy-C₁-C₆-alkyl such as hydroxymethyl. Presently preferred is the case where R^{8c} is —OH. Each Ar² is an aryl, heteroaryl or cycloalkyl ring and may be, for example, any of those aryl, heteroaryl, C₁-C₆-alkyl, or cycloalkyl rings specifically mentioned in the discussion of R⁵ above. Preferred Ar² rings include phenyl. The bridge -Q between the two Ar² rings is —O—, —CH₂— or —CH₂CH₂—.

Of the R⁴ options (a), (b), (c) and (d), it is presently preferred that R⁴ be a group (a) or (b) or (c).

A preferred subclass of compounds with which the invention is concerned consists of those of formula (IA)

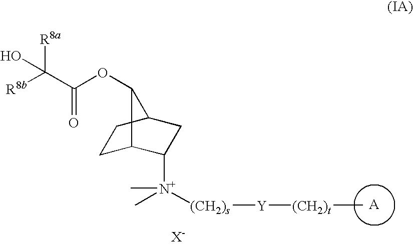

wherein ring A is an optionally substituted phenyl ring, or a monocyclic heterocyclic ring of 5 or 6 ring atoms, or a phenyl-fused-heterocycloalkyl ring system wherein the heterocycloalkyl ring is a monocyclic heterocyclic ring of 5 or 6 ring atoms; R^{8a} is phenyl, thienyl, cyclopentyl or cyclohexyl; R^{8b} is phenyl; thienyl, cyclopentyl or cyclohexyl; s is 1, 2, 3, 4, 5, 6 or 7 and t is 0, 1, 2, 3, 4, 5, 6 or 7 provided that s+t is not greater than 10; Y is a bond or —O—, and X⁻ is a pharmaceutically acceptable anion. In compounds (IA), it is currently preferred that ring A is optionally substituted phenyl, wherein optional substituents are selected from alkoxy, halo especially fluoro or chloro, C₁-C₃-alkyl, amino C₁-C₃-acyl, amino C₁-C₃-alkyl, and aminosulfonyl.

Another preferred subclass of compounds with which the invention is concerned consists of those of formula (IB)

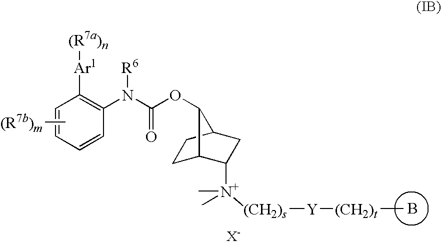

wherein ring B is an optionally substituted phenyl ring or a monocyclic heterocyclic ring of 5 or 6 ring atoms or an aryl-fused heterocycloalkyl ring; s is 1, 2, 3, 4, 5, 6 or 7 and t is 0, 1, 2, 3, 4, 5, 6 or 7 provided that s+t is not greater than 10; Y is a bond or —O—; and R⁶, Ar¹, R^{7a} and R^{7b} are as defined for group (a) above; and X⁻ is a pharmaceutically acceptable anion. In compounds (IB), it is currently preferred that ring B is (i) optionally substituted phenyl, wherein optional substituents are selected from alkoxy, halo especially fluoro or chloro, C₁-C₃-alkyl, amino C₁-C₃-acyl, amino C₁-C₃-alkyl, and aminosulfonyl;

Another preferred subclass of compounds with which the invention is concerned consists of those of formula (IC)

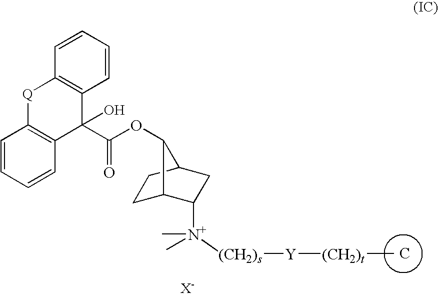

wherein ring C is an optionally substituted phenyl ring or a monocyclic heterocyclic ring of 5 or 6 ring atoms or an aryl-fused heterocycloalkyl ring; Q is an oxygen atom, —CH₂—, —CH₂CH₂— or a bond; s is 1, 2, 3, 4, 5, 6 or 7 and t is 0, 1, 2, 3, 4, 5, 6 or 7 provided that s+t is not greater than 10 and Y is a bond or —O—; and X⁻ is a pharmaceutically acceptable anion. In compounds (IC), it is currently preferred that ring C is optionally substituted phenyl, wherein optional substituents are selected from alkoxy, halo especially fluoro or chloro, C₁-C₃-alkyl, amino C₁-C₃-acyl, amino C₁-C₃-alkyl, and aminosulfonyl;

In subclasses (IA), (IB) and (IC), s+t may be, for example 1, 2, 3, 4, 5, 6, or 7 and may arise from suitable combinations of t and s such as where t is 0, 1, 2, 3, 4, 5 or 6 and s is 1, 2, 3, 4, 5, 6 or 7. In compounds (IA), (IB) and (IC), a currently preferred combination of t, Y and s is where t is 0, s is 3, and Y is —O—. A further currently preferred combination is where Y is a bond and s+t is 2, 3 or 4.

In subclasses (IA), (IB) and (IC) as in the compounds of the invention generally, compounds predominantly in the anti-endo configuration are preferred.

Examples of compounds of the invention include those of the Examples herein.

The present invention is also concerned with pharmaceutical formulations comprising, as an active ingredient, a compound of the invention. Other compounds may be combined with compounds of this invention for the prevention and treatment of inflammatory diseases of the lung. Thus the present invention is also concerned with pharmaceutical compositions for preventing and treating respiratory-tract disorders such as chronic obstructive lung disease, chronic bronchitis, asthma, chronic respiratory obstruction, pulmonary fibrosis, pulmonary emphysema, and allergic rhinitis comprising a therapeutically effective amount of a compound of the invention and one or more other therapeutic agents.

Other compounds may be combined with compounds of this invention for the prevention and treatment of inflammatory diseases of the lung. Accordingly the invention includes a combination of an agent of the invention as hereinbefore described with one or more anti-inflammatory, bronchodilator, antihistamine, decongestant or anti-tussive agents, said agents of the invention hereinbefore described and said combination agents existing in the same or different pharmaceutical compositions, administered separately or simultaneously. Preferred combinations would have two or three different pharmaceutical compositions. Suitable therapeutic agents for a combination therapy with compounds of the invention include:

One or more other bronchodilators such as PDE3 inhibitors;
Methyl xanthines such as theophylline;
Other muscarinic receptor antagonists;
A corticosteroid, for example fluticasone propionate, ciclesonide, mometasone furoate or budesonide, or steroids described in WO02/88167, WO02/12266, WO02/100879, WO02/00679, WO03/35668, WO03/48181, WO03/62259, WO03/64445, WO03/72592, WO04/39827 and WO04/66920;
A non-steroidal glucocorticoid receptor agonist;
A β2-adrenoreceptor agonist, for example albuterol (salbutamol), salmeterol, metaproterenol, terbutaline, fenoterol, procaterol, carmoterol, indacaterol, formoterol, arformoterol, picumeterol, GSK-159797, GSK-597901, GSK-159802, GSK-64244, GSK-678007, TA-2005 and also compounds of EP1440966, JP05025045, WO93/18007, WO99/64035, US2002/0055651, US2005/0133417, US2005/5159448, WO00/075114, WO01/42193, WO01/83462, WO02/66422, WO02/70490, WO02/76933, WO03/24439, WO03/42160, WO03/42164, WO03/72539, WO03/91204, WO03/99764, WO04/16578, WO04/016601, WO04/22547, WO04/32921, WO04/33412, WO04/37768, WO04/37773, WO04/37807, WO0439762, WO04/39766, WO04/45618, WO04/46083, WO04/71388, WO04/80964, EP1460064, WO04/087142, WO04/89892, EP01477167, US2004/0242622, US2004/0229904, WO04/108675, WO04/108676, WO05/033121, WO05/040103, WO05/044787, WO04/071388, WO05/058299, WO05/058867, WO05/065650, WO05/066140, WO05/070908, WO05/092840, WO05/092841, WO05/092860, WO05/092887, WO05/092861, WO05/090288, WO05/092087, WO05/080324, WO05/080313, US20050182091, US20050171147, WO05/092870, WO05/077361, DE10258695, WO05/111002, WO05/111005, WO05/110990, US2005/0272769 WO05/110359, WO05/121065, US2006/0019991, WO06/016245, WO06/014704, WO06/031556, WO06/032627, US2006/0106075, US2006/0106213, WO06/051373, WO06/056471;
A leukotriene modulator, for example montelukast, zafirlukast or pranlukast;
protease inhibitors, such as inhibitors of matrix metalloprotease for example MMP12 and TACE inhibitors such as marimastat, DPC-333, GW-3333;
Human neutrophil elastase inhibitors, such as sivelestat and those described in WO04/043942, WO05/021509, WO05/021512, WO05/026123, WO05/026124, WO04/024700, WO04/024701, WO04/020410, WO04/020412, WO05/080372, WO05/082863, WO05/082864, WO03/053930;
Phosphodiesterase-4 (PDE4) inhibitors, for example roflumilast, arofylline, cilomilast, ONO-6126 or IC-485;
Phosphodiesterase-7 inhibitors;
An antitussive agent, such as codeine or dextramorphan;
Kinase inhibitors, particularly P38 MAPKinase inhibitors;
P2X7 anatgonists;
iNOS inhibitors;
A non-steroidal anti-inflammatory agent (NSAID), for example ibuprofen or ketoprofen;
A dopamine receptor antagonist;
TNF-α inhibitors, for example anti-TNF monoclonal antibodies, such as Remicade and CDP-870 and TNF receptor immunoglobulin molecules, such as Enbrel;
A2a agonists such as those described in EP1052264 and EP1241176;
A2b antagonists such as those described in WO2002/42298;
Modulators of chemokine receptor function, for example antagonists of CCR1, CCR2, CCR3, CXCR2, CXCR3, CX3CR1 and CCR8, such as SB-332235, SB-656933, SB-265610, SB-225002, MCP-1(9-76), RS-504393, MLN-1202, INCB-3284;
Compounds which modulate the action of prostanoid receptors, for example a PGD₂ (DP1 or CRTH2), or a thromboxane A₂ antagonist eg ramatroban;
Compounds which modulate Th1 or Th2 function, for example, PPAR agonists; Interleukin 1 receptor antagonists, such as Kineret;
Interleukin 10 agonists, such as Ilodecakin;
HMG-CoA reductase inhibitors (statins); for example rosuvastatin, mevastatin, lovastatin, simvastatin, pravastatin and fluvastatin;
Mucus regulators such as INS-37217, diquafosol, sibenadet, CS-003, talnetant, DNK-333, MSI-1956, gefitinib;
Antiinfective agents (antibiotic or antiviral), and antiallergic drugs including, but not limited to, anti-histamines.

The weight ratio of the first and second active ingredients may be varied and will depend upon the effective dose of each ingredient. Generally, an effective dose of each will be used.

Any suitable route of administration may be employed for providing a mammal, especially a human, with an effective dosage of a compound of the present invention. In therapeutic use, the active compound may be administered by any convenient, suitable or effective route. Suitable routes of administration are known to those skilled in the art, and include oral, intravenous, rectal, parenteral, topical, ocular, nasal, buccal and pulmonary.

The magnitude of prophylactic or therapeutic dose of a compound of the invention will, of course, vary depending upon a range of factors, including the activity of the specific compound that is used, the age, body weight, diet, general health and sex of the patient, time of administration, the route of administration, the rate of excretion, the use of any other drugs, and the severity of the disease undergoing treatment. In general, the daily dose range for inhalation will lie within the range of from about 0.1 μg to about 10 mg per kg body weight of a human, preferably 0.1 μg to about 0.5 mg per kg, and more preferably 0.1 μg to 50 μg per kg, in single or divided doses. On the other hand, it may be necessary to use dosages outside these limits in some cases. Compositions suitable for administration by inhalation are known, and may include carriers and/or diluents that are known for use in such compositions. The composition may contain 0.01-99% by weight of active compound. Preferably, a unit dose comprises the active compound in an amount of 1 μg to 10 mg. For oral administration suitable doses are 10 μg per kg to 100 mg per kg, preferably 40 μg per kg to 4 mg per kg.

Another aspect of the present invention provides pharmaceutical compositions which comprise a compound of the invention and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the invention, additional active ingredient(s), and pharmaceutically acceptable excipients.

The pharmaceutical compositions of the present invention comprise a compound of the invention as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids, and salts of quaternary ammonium compounds with pharmaceutically acceptable counter-ions.

For delivery by inhalation, the active compound is preferably in the form of microparticles. They may be prepared by a variety of techniques, including spray-drying, freeze-drying and micronisation.

By way of example, a composition of the invention may be prepared as a suspension for delivery from a nebuliser or as an aerosol in a liquid propellant, for example for use in a pressurised metered dose inhaler (PMDI). Propellants suitable for use in a PMDI are known to the skilled person, and include CFC-12, HFA-134a, HFA-227, HCFC-22 (CCl₂F₂) and HFA-152 (C₂H₄F₂) and isobutane.

In a preferred embodiment of the invention, a composition of the invention is in dry powder form, for delivery using a dry powder inhaler (DPI). Many types of DPI are known.

Microparticles for delivery by administration may be formulated with excipients that aid delivery and release. For example, in a dry powder formulation, microparticles may be formulated with large carrier particles that aid flow from the DPI into the lung. Suitable carrier particles are known, and include lactose particles; they may have a mass median aerodynamic diameter of greater than 90 μm.

In the case of an aerosol-based formulation, an example is:
Compound of the invention 24 mg/canister
Lecithin, NF Liq. Conc. 1.2 mg/canister
Trichlorofluoromethane, NF 4.025 g/canister
Dichlorodifluoromethane, NF 12.15 g/canister.

The active compounds may be dosed as described depending on the inhaler system used. In addition to the active compounds, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321). Additionally, compounds of the invention may be delivered in multi-chamber devices thus allowing for delivery of combination agents.

### Methods of Synthesis

The compounds of the invention can be prepared according to the procedures of the following schemes and examples, using appropriate materials, and are further exemplified by the following specific examples. Moreover, by utilising the procedures described with the disclosure contained herein, one of ordinary skill in the art can readily prepare additional compounds of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention may be isolated in the form of their pharmaceutically acceptable salts, such as those described previously herein.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 1999, may be used.

Compounds of the invention may be prepared according to the routes illustrated in Scheme 1.

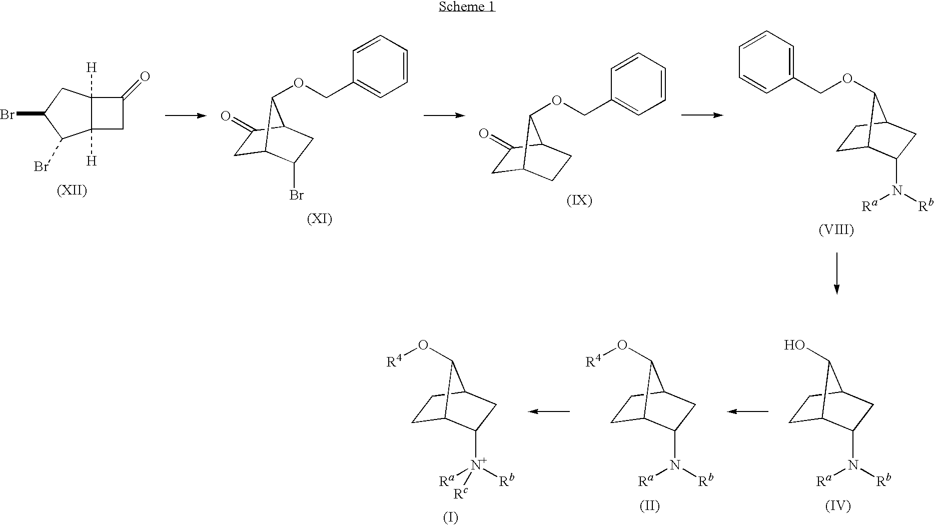

Compounds of general formula (I) wherein R^{a}, R^{b} and R^{c} and R⁴ are as defined for R¹, R², R³ and R⁴ in compounds of formula (I) can be prepared from compounds of general formula (II):

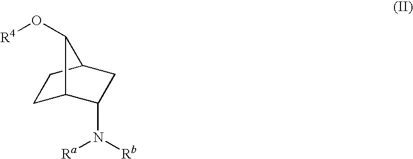

by reaction with a compound of general formula (III):

R^{c}-X (III)

wherein X is a leaving group such as halogen, tosylate, mesylate. The reaction can be performed in a range of solvents, such as acetonitrile, chloroform, DMF or DMSO, at a temperature from 0° C. to the reflux temperature of the solvent, preferably from ambient temperature to the reflux temperature of the solvent.

Compounds of general formula (I) exist in two enantiomeric forms which can be prepared in homochiral form by starting with homochiral (XII). Alternatively, chiral separation of racemic material by chiral hplc may be undertaken.

Compounds of general formula (II) wherein R⁴ is the group of formula (a), as defined above and R⁶ is H may be prepared from compounds of general formula (IV):

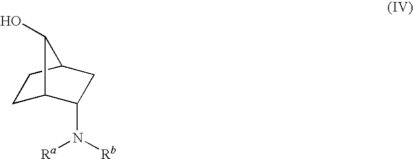

wherein R^{a} and R^{b} are as defined above, by reaction with a compound of general formula (V):

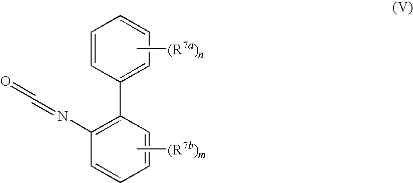

wherein R^{7a}, R^{7b}, n and m are as defined for general formula (I). The reaction may take place in a range of non-nucleophilic organic solvents such as DMF or toluene at a range of temperatures, preferably between 0° C. and the reflux temperature of the solvent.

Compounds of general formula (V) are well known in the art and are readily available or can be prepared by known methods.

Compounds of general formula (II) in which R⁴ is the group of formula (b) as defined above, may be prepared from compounds of general formula (IV) by reaction with a compound of general formula (VI):

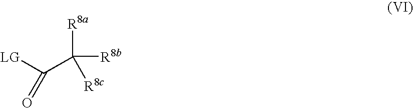

wherein R^{8a}, R^{8b}, and R^{8c} are as defined for general formula (I) and LG is a leaving group, for example, an O-alkyl, halogen or 1-imidazolyl group. The reaction is conducted in the presence of a strong base such as NaH in a solvent such as toluene, THF or dichloromethane at a range of temperatures, preferably between 0° C. and the reflux temperature of the solvent.

Compounds of general formula (VI) wherein R^{8a}, R^{8b}, and R^{8c} are as defined for general formula (I) and Y is an O-alkyl, halogen or 1-imidazolyl group can be prepared from compounds of general formula (VII) by known methods.

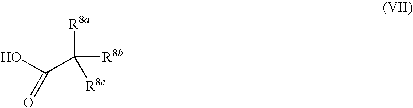

Compounds of general formula (VII) are well known in the art and are readily available or can be prepared by known methods such as those described in WO01/04118.

Compounds of general formula (II) in which R⁴ is the group of formula (c) as defined above, may be prepared from compounds of general formula (IV) by reaction with a compound of general formula (VIa):

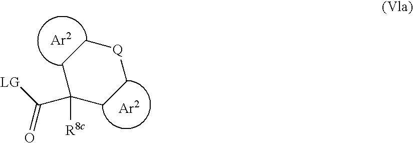

wherein Ar², R^{8c} Q and LG are as defined above, using conditions similar to those employed for the preparation of compounds of formula (II) by reaction of compounds of formula (IV) with compounds of formula (VI) above.

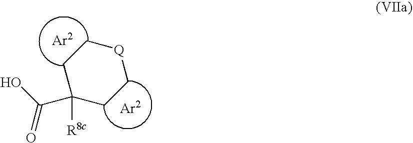

Compounds of formula (VIa) may be prepared from compounds of formula (VIIa) by methods analogous to those used for the preparation of compounds of formula (VI) from compounds of formula (VII) above.

Compounds of general formula (VII) are well known in the art and are readily available or can be prepared by known methods.

Compounds of general formula (IV) can be prepared from compounds of general formula (VIII):

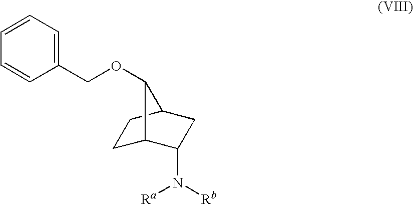

by reaction with hydrogen in the presence of a catalyst, preferably palladium hydroxide on carbon, in a polar solvent such as MeOH or EtOAc, optionally in the presence of a protic acid such as sulphuric or acetic acid.

Compounds of general formula (VIII) can be prepared from compounds of general formula (IX):

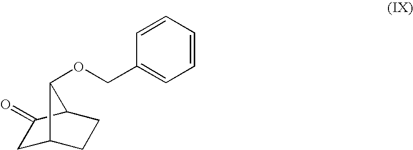

by reaction with an amine of general formula (X):

R^{a}R^{b}NH (X)

in the presence of a reducing agent such as sodium triacetoxyborohydride in a polar solvent such as THF or DCE, optionally in the presence of acetic acid.

Compounds of general formula (IX) can be prepared from compounds of general formula (XI):

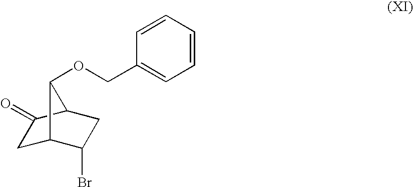

by reaction with a tin reagent, preferably Bu₃SnH and a radical initiator, preferably AIBN. The reaction can be performed in a range of solvents, preferably toluene, at a range of temperatures, preferably between room temperature and the reflux temperature of the solvent.

Compounds of general formula (XI) can be prepared from compounds of general formula (XII):

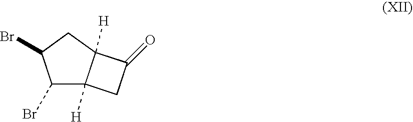

by reaction with benzyl alcohol. The reaction is performed in the presence of a strong base such as NaH in a range of solvents, preferably THF or DMF at a range of temperatures, preferably between −78° C. and ambient temperature.

Compounds of formula (XII) are known in the art and can be prepared from homochiral starting bicycle[3.2.0]heptenone by bromination (Synthesis (1977), 155-166). The resolution of the bicycloheptenone is described in EP0074856.

The following non-limiting Examples illustrate the invention.

### GENERAL EXPERIMENTAL DETAILS

All reactions were carried out under an atmosphere of nitrogen unless specified otherwise.

Where products were purified by column chromatography, ‘flash silica’ refers to silica gel for chromatography, 0.035 to 0.070 mm (220 to 440 mesh) (e.g. Fluka silica gel 60), and an applied pressure of nitrogen up to 10 p.s.i accelerated column elution. Where thin layer chromatography (TLC) has been used, it refers to silica gel TLC using plates, typically 3×6 cm silica gel on aluminium foil plates with a fluorescent indicator (254 nm), (e.g. Fluka 60778). All solvents and commercial reagents were used as received.

All compounds containing a basic centre(s), which were purified by HPLC, were obtained as the TFA salt unless otherwise stated.

### LC-MS Method 1

Waters Micromass ZQ with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow ml/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection - MS, ELS, UV (100 μl split to MS with in-line UV detector)
MS ionisation method - Electrospray (positive and negative ion)

LC-MS Method 2

Waters Micromass ZQ with a C18-reverse-phase column (Higgins Clipeus 5 micron C18 100×3.0 mm or equivalent), elution with A: water+0.1% formic acid; B: acetonitrile+0.1% formic acid. Gradient:

| Gradient - Time | flow ml/min | % A | % B |
|---|---|---|---|
| 0.00 | 1.0 | 95 | 5 |
| 1.00 | 1.0 | 95 | 5 |
| 15.0 | 1.0 | 5 | 95 |
| 20.0 | 1.0 | 5 | 95 |
| 22.0 | 1.0 | 95 | 5 |
| 25.0 | 1.0 | 95 | 5 |

Detection - MS, ELS, UV (100 μl split to MS with in-line UV detector)
MS ionisation method - Electrospray (positive and negative ion)

Abbreviations used in the experimental section:DCM=dichloromethane; EtOH=ethanol; DIPEA=di-isopropylethylamine; EDCl=1-(3-dimethylaminopropyl)-3-ethylcarbodiimide hydrochloride; DMAP=dimethylaminopyridine; RT=room temperature; HATU=O-(7-Azabenzotriazol-1-yl)-N,N,N′,N′-tetramethyluroniumhexafluorophosphate; TFA=trifluoroacetic acid; Rt=retention time.

### Example 1

### Anti (±)-biphenyl-2-yl-carbamic acid 2-dimethylamino-bicyclo[2.2.1]hept-7-yl ester. (II): R^{a}, R^{b}═CH₃, R⁴=biphenyl-2-ylcarbamyl

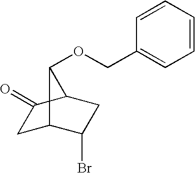

### a. (±)-7-Benzyloxy-5-bromo-bicyclo[2.2.1]heptan-2-one. (XI)

To a suspension of NaH (60% dispersion in oil; 433 mg, 10.8 mmol) in dry THF (7 mL) under N₂ was added dropwise benzyl alcohol (1.07 mL, 10.4 mmol). The mixture was stirred for 30 mins then cooled to −30° C. and treated dropwise with a solution of (±)-2,3-dibromo-bicyclo[3.2.0]heptan-6-one (1.32 g, 4.9 mmol) in dry THF (6 mL) and the reaction mixture was allowed to warm up to 0° C. over 2 h. The tan coloured heterogeneous mixture was diluted with ethyl acetate and then washed with 10% aqueous citric acid solution, water and brine, dried over magnesium sulfate and evaporated. The crude product was purified by silica gel chromatography eluting with 15% ether/pentane to give the title compound as a colourless syrup (609 mg). ¹H NMR (CDCl₃, 400 MHz): δ 1.79 (1H, ddd, J=14.3 Hz, 4.2 Hz, 1.5 Hz), 2.15 (1H, m), 2.65 (1H, m), 2.80 (1H, m), 2.82 (1H, d, J=19.1 Hz), 2.86 (1H, m), 4.03 (1H, q, J=1.8 Hz), 4.50 (1H, d, J=11.7 Hz), 4.53 (1H, d, J=11.7 Hz), 4.71 (1H, m), 7.27-7.40 (5H, m).

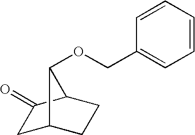

### b. (±)-7-Benzyloxy-bicyclo[2.2.1]heptan-2-one. (IX)

To a mixture of (±)-7-benzyloxy-5-bromo-bicyclo[2.2.1]heptan-2-one (609 mg, 2.06 mmol) and AIBN (34 mg, 0.21 mmol) in 30 mL dry degassed toluene was added dropwise tributyltin hydride (0.72 mL, 2.68 mmol) and the solution was heated to 80° C. for 1.5 h. The reaction mixture was concentrated under reduced pressure, adsorbed onto diatomaceous earth and chromatographed eluting with 1-15% ether/pentane to give the title compound as a colourless gum (446 mg). ¹H NMR (CDCl₃, 400 MHz): δ 1.49 (2H, m), 1.99 (1H, d, J=18.5 Hz), 2.10 (3H, m), 2.62 (1H, m), 2.67 (1H, m), 3.91 (1H, s), 4.51 (1H, d, J=11.7 Hz), 4.53 (1H, d, J=11.7 Hz), 7.27-7.39 (5H, m).

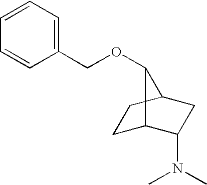

### c. (±)-(7-Benzyloxy-bicyclo[2.2.1]hept-2-yl)-dimethyl-amine. (VIII): R^{a}, R^{b}═CH₃; R⁴=Bn

To a solution of (±)-7-benzyloxy-bicyclo[2.2.1]heptan-2-one (262 mg, 1.21 mmol) and dimethylamine (2M solution in THF, 1.21 mL, 2.42 mmol) in 2 mL dry DCE was added acetic acid (69 μL, 1.21 mmol) followed by sodium triacetoxyborohydride (385 mg, 1.82 mmol) and a small amount of 3 Å molecular sieves. The mixture was stirred at ambient temperature for 6 h. The reaction was quenched with 10 mL saturated aqueous sodium bicarbonate solution and stirred for 10 mins. The reaction mixture was extracted twice with EtOAc and the combined organic phase was washed with water and brine, dried over anhydrous sodium sulfate and evaporated to give the title compound as a pale yellow oil (259 mg), which was used without further purification. LC-MS (Method 1): Rt 1.73 min, m/z 246.21 [MH+].

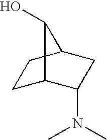

### d. (±)-2-Dimethylamino-bicyclo[2.2.1]heptan-7-ol. (IV): R^{a}, R^{b}═CH₃

A solution of (±)-(7-benzyloxy-bicyclo[2.2.1]hept-2-yl)-dimethylamine (236 mg, 0.96 mmol) in dry MeOH (8 mL) was added to 20 wt % palladium hydroxide on carbon (50 mg). The reaction vessel was evacuated and purged with nitrogen three times and then a 25% solution of sulfuric acid in MeOH (320 μL, 1.5 mmol) was added. The reaction was stirred under an atmosphere of hydrogen (hydrogen balloon) for 20 h. The reaction was quenched with 200 mg solid sodium carbonate and 3 mL water and evaporated. The residue was partitioned between DCM and brine and the layers separated. The aqueous phase was extracted four more times with DCM and the combined organic phase was dried over sodium sulfate and evaporated to give a 3:1 mixture of the title compound and recovered starting material as a colourless oil that was used without further purification (105 mg).

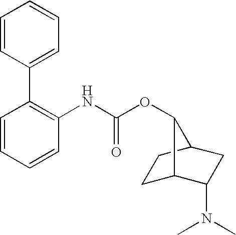

### e. (±) Anti-biphenyl-2-yl-carbamic acid 2-dimethylamino-bicyclo[2.2.1]hept-7-yl ester, (II): R^{a}, R^{b}═CH₃; R⁴=biphenyl-2-carbamyl

A solution of a 3:1 mixture of (±)-2-dimethylamino-bicyclo[2.2.1]heptan-7-ol and (±)-(7-benzyloxy-bicyclo[2.2.1]hept-2-yl)-dimethyl-amine (96 mg) in dry toluene (2 mL) was treated with 2-biphenyl isocyanate (145 mg, 0.74 mmol) and the mixture was heated at 80° C. for 2.5 h. The volatiles were evaporated and the residue was purified by silica gel chromatography eluting with 2-6% MeOH/DCM to give the title compound as a colourless syrup (125 mg). LC-MS (Method 1): Rt 7.05 min, m/z 351.16 [MH+]. LC-MS (Method 1): Rt 2.01 and 2.09 min, m/z 351.27 [MH+]. ¹H NMR (CDCl₃, 400 MHz): δ 0.99 (1H, dd, J=12.7 Hz, 4.9 Hz), 1.34 (1H, m), 1.51 (1H, m), 1.73 (1H, m), 1.83 (2H, m), 2.13 (6H, s), 2.20 (2H, m), 2.39 (1H, m), 4.72 (1H, s), 6.57 (1H, s, br), 7.13 (1H, td, J=7.5, 1.2 Hz), 7.22 (1H, dd, J=7.5, 1.7 Hz), 7.36 (3H, m), 7.40 (1H, m), 7.48 (2H, m), 8.06 (1H, s, br).

### Example 2

### a. Anti (1S,2S) hydroxy-di-thiophen-2-yl-acetic acid 2-[methyl-(3-phenyl-propyl)-amino]-bicyclo[2.2.1]hept-7-yl ester. (II): R^{a}═CH₃; R^{b}=3-phenyl-1-propyl; R⁴=hydroxyl-dithiophen-2-ylcarbonyl

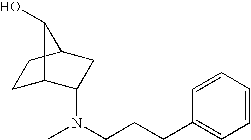

### b. Anti (1S,2S) 2-[methyl-(3-phenyl-propyl)-amino]-bicyclo[2.2.1]heptan-7-ol, (IV): R^{a}═CH₃; R^{b}=3-phenyl-1-propyl

The title compound was prepared from (1S,2R,3R)-2,3-dibromo-bicyclo[3.2.0]heptan-6-one and methyl-(3-phenylpropyl)-amine using analogous methods to those in Example 1. ¹H NMR (CDCl₃, 400 MHz): δ 0.91-0.95 (1H, m), 1.32-1.38 (1H, m), 1.54-1.62 (2H, m), 1.68-1.89 (6H, m), 1.98 (1H, m), 2.08 (3H, m), 2.26-2.29 (3H, m), 2.59-2.63 (2H, m), 3.97 (1H, m), 7.15-7.19 (3H, m), 7.25-7.29 (2H, m).

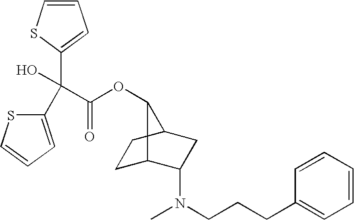

### c. Anti (1S,2S) hydroxy-di-thiophen-2-yl-acetic acid 2-[methyl-(3-phenyl-propyl)-amino]-bicyclo[2.2.1]hept-7-yl ester. (II): R^{a}═CH₃; R^{b}=3-phenyl-1-propyl; R⁴=hydroxyl-dithiophen-2-ylcarbonyl

A solution of anti (1S,2S) 2-[methyl-(3-phenyl-propyl)-amino]bicyclo[2.2.1]heptan-7-ol (165 mg, 0.63 mmol) in dry toluene (10 mL) was stirred at ambient temperature under a nitrogen atmosphere with sodium hydride (76 mg of a 60% dispersion in mineral oil). When gas evolution had subsided, hydroxy-di-thiophen-2-yl-acetic acid ethyl ester (205 mg, 0.76 mmol) was added portion wise over 5 minutes before the reaction was immediately placed in a preheated oil bath at 80° C. After 1 h the reaction was allowed to cool before being quenched with sat. ammonium chloride solution. The reaction was extracted into ethyl acetate and the combined organic extracts dried over MgSO₄ and concentrated in vacuo. Purification by chromatography using 0-3% methanol-CH₂Cl₂ as eluent gave the title compound as a gum (127 mg). LC-MS (Method 2): Rt 7.90 min, m/z 482 [MH]+. ¹H NMR (CDCl₃, 400 MHz): δ 0.88-1.01 (1H, m), 1.21-1.34 (2H, m), 1.46-1.59 (1H, m), 1.68-1.87 (4H, m), 2.01-2.09 (3H, s), 2.18-2.27 (3H, m), 2.30-2.37 (2H, m), 2.54-2.60 (2H, m), 4.75-4.97 (1H, m), 4.83 (1H, m), 6.93-6.97 (2H, m), 7.12-7.18 (5H, m), 7.22-7.28 (4H, m).

### Example 3

### Anti (1S,2S) [7-(2-hydroxy-2,2-di-thiophen-2-yl-acetoxy)-bicyclo[2.2.1]hept-2-yl]-dimethyl-(3-phenyl-propyl)-ammonium bromide. (I): R^{a}, R^{b}═CH₃; R^{b}=3-phenyl-1-propyl; R⁴=hydroxyl-dithiophen-2-ylcarbonyl

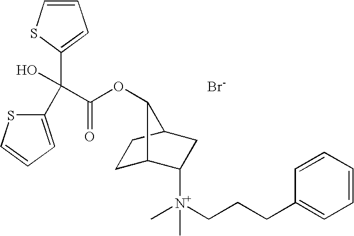

A solution of anti (1S,2S) hydroxy-di-thiophen-2-yl-acetic acid 2-[methyl-(3-phenyl-propyl)-amino]-bicyclo[2.2.1]hept-7-yl ester (33 mg, 0.069 mmol) in a 30% w/w solution of methyl bromide in acetonitrile (1 mL) was heated in a sealed tube for 2 days at 50° C. The solvent was removed and purification by chromatography using 0-10% methanol-CH₂Cl₂ as eluent gave the title compound as a foam (33 mg). LC-MS (Method 2): Rt 7.45 min, m/z 496 [M]+; ¹H NMR (CDCl₃, 400 MHz) δ 1.33-1.41 (1H, m), 1.45-1.56 (1H, m), 1.57-1.67 (2H, m), 1.69-1.79 (1H, m), 2.04-2.21 (3H, m), 2.43 (1H, m), 2.64-2.79 (3H, m), 3.23 (3H, s), 3.25 (3H, s), 3.43-3.55 (1H, m), 3.56-3.68 (1H, m), 4.31 (1H, m), 4.89-4.92 (1H, m), 5.02-5.05 (1H, m), 6.93-6.99 (2H, m), 7.10-7.30 (9H, m).

The following examples were prepared in a similar manner to that described for examples 1-3.

| | | | Rt/min |
|---|---|---|---|
| | | | (Method 2); |
| | | | [MH]+ or |
| Name | Structure | ]hu 1]l H NMR (400 MHz) | [M]+ |
| Anti (1R, 2R) hydroxy-di-thiophen- 2-yl-acetic acid 2- [methyl-(3-phenyl- propyl)-amino]- bicyclo[2.2.1]hept-7- yl ester | 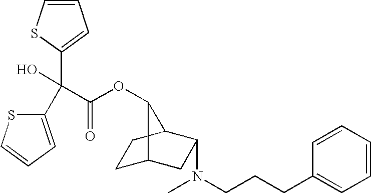 | (CD₃OD) 0.83-0.93 (1H, m), 1.15-1.30 (2H, m), 1.43-1.54 (1H, m), 1.60-1.81 (4H, m), 2.06 (3H, m), 2.10-2.15 (1H, m), 2.19-2.30 (3H, m), 2.33- 2.40 (1H, m), 2.51-2.59 (2H, m), 4.75-4.78 (1H, m), 4.78- 4.83 (1H, m), 6.90-6.98 (2H, m), 7.06-7.25 (7H, m), 7.29- 7.36 (2H, m). | 7.29; 482 |
| Anti (1R, 2R) [7-(2- hydroxy-2,2-di- thiophen-2-yl- acetoxy)- bicyclo[2.2.1]hept-2- yl]-dimethyl-(3- phenyl-propyl)- ammonium bromide | 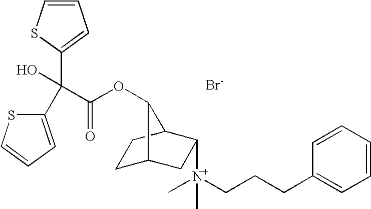 | (CDCl₃) 1.33-1.42 (1H, m), 1.44-1.57 (1H, m), 1.58-1.80 (2H, m), 1.81-1.87 (1H, m), 2.06-2.21 (3H, m), 2.43-2.48 (1H, m), 2.65-2.81 (3H, m), 3.27 (3H, s), 3.29 (3H, s), 3.44-3.59 (1H, m), 3.59-3.71 (1H, m), 4.24-4.34 (1H, m), 4.87-4.92 (2H, m), 6.93-6.99 (2H, m), 7.11-7.30 (9H, m). | 7.95; 496 |
| Anti (1S, 2S) (7- benzyloxybi- cyclo[2.2.1]hept-2- yl)-dimethyl-(3- phenyl-propyl)- ammonium bromide | 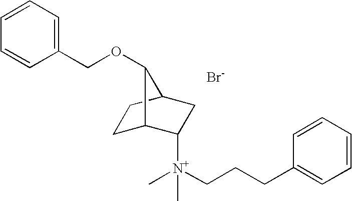 | (CDCl₃) 1.28-1.36 (1H, m), 1.43-1.52 (1H, m), 1.65-1.74 (1H, m), 1.9-2.2 (5H, m), 2.33-2.38 (1H, m), 2.61-2.66 (1H, m), 2.72-2.79 (2H, m), 3.26 (3H, s), 3.29 (3H, s), 3.54-3.66 (2H, m), 3.87 (1H, m), 4.23-4.32 (1H, m), 4.46 (2H, s), 7.18-7.37 (10H). | 7.37; 364 |
| Anti (1S, 2S) Hydroxy-di-thiophen- 2-yl-acetic acid 2-[methyl-(3- phenoxy-propyl)- amino]-bicyclo[2.2.1] hept-7-yl ester | 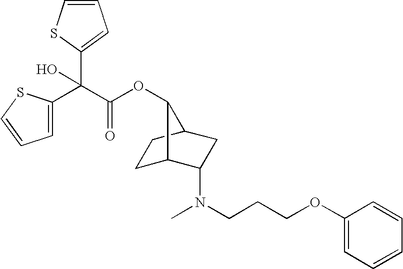 | (CDCl₃) 0.94-1.00 (1H, m), 1.12-1.20 (1H, m), 1.20-1.30 (1H m), 1.43-1.54 (1H, m), 1.73-1.84 (2H, m), 1.84-1.92 (2H, m), 2.06 (3H, s), 2.16- 2.21 (1H, m), 2.28-2.48 (4H, m), 3.92-4.04 (2H, m), 4.74 (1H, s), 4.84 (1H, s), 6.84- 6.96 (5H, m), 7.12-7.15 (2H, m), 7.22-7.27 (4H, m). | 7.97; 498 |
| Anti (1S, 25) [7-(2- Hydroxy-2,2-di- thiophen-2-yl- acetoxy)- bicyclo[2.2.1]hept-2- yl]-dimethyl- (3-phenoxy- propyl)-ammonium bromide | 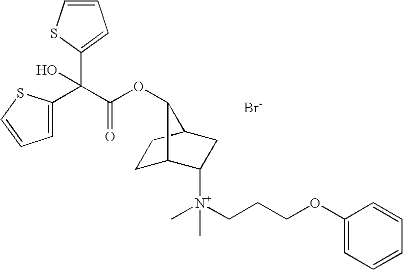 | (CDCl₃) 1.37-1.45 (1H, m), 1.56-1.73 (3H, m), 1.79-1.87 (1H, m), 2.20-2.37 (3H, m), 2.48-2.52 (1H, m), 2.88-2.92 (1H, m), 3.30-3.35 (6H, m), 3.74-3.90 (2H, m), 4.06-4.11 (2H, m), 4.26-4.34 (1H, m), 4.77 (1H, s), 4.93 (1H, s), 6.81-6.85 (2H, m), 6.93-6.97 (3H, m), 7.11-7.13 (2H, m), 7.22-7.28 (4H, m). | 8.07; 512 |
| Anti (1S, 2S) Hydroxy-di-thiophen- 2-yl-acetic acid 2-[methyl-(4- phenyl-butyl)-amino]- bicyclo[2.2.1]hept-7- yl ester | 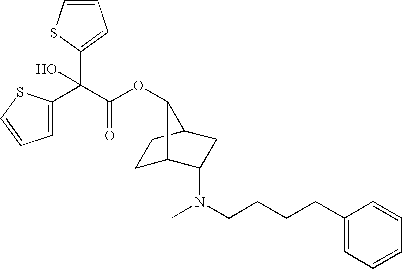 | (CDCl₃) 0.95-1.00 (1H, m), 1.23-1.33 (2H, m), 1.39-1.65 (5H, m), 1.73-1.85 (2H, m), 2.01 (3H, s), 2.15-2.24 (3H, m), 2.29-2.38 (2H, m), 2.56- 2.61 (2H, m), 4.75 (1H, s), 4.84 (1H, s), 6.93-6.97 (2H, m), 7.12-7.17 (5H, m), 7.22- 7.27 (4H, m). | 8.27; 496 |
| Anti (1S, 2S) [7-(2- Hydroxy-2,2-di- thiophen-2-yl- acetoxy)- bicyclo[2.2.1]hept-2- yl]- dimethyl-(4-phenyl- butyl)-ammonium bromide | 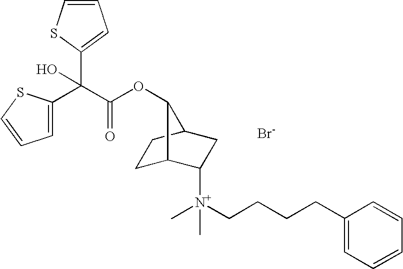 | (CDCl₃) 1.35-1.43 (1H, m), 1.50-1.80 (8H, m), 2.10-2.20 (1H, m), 2.45-2.49 (1H, m), 2.63-2.70 (2H, m), 2.73-2.77 (1H, m), 3.24 (6H, s), 3.47- 3.58 (2H, m), 4.18-4.25 (1H, m), 4.73 (1H, s), 4.87 (1H, s), 6.93-6.97 (2H, m), 7.10-7.17 (5H, m), 7.21-7.28 (4H, m). | 8.40; 510 |
| Anti (1S, 2S) Hydroxy-di-thiophen- 2-yl-acetic acid 2-(methyl- phenethyl-amino)- bicyclo[2.2.1 ] hept-7-yl ester | 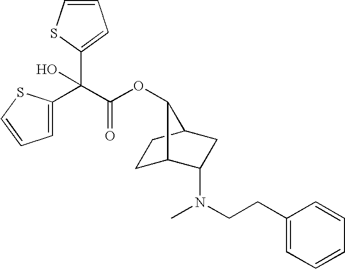 | (CDCl₃) 0.89-0.94 (1H, m), 1.16-1.33 (2H, m), 1.44-1.55 (1H, m), 1.72-1.82 (2H, m), 2.16 (3H, s), 2.17-2.21 (1H, m), 2.35-2.39 (1H, m), 2.40- 2.50 (3H, m), 2.63-2.76 (2H, m), 4.74 (1H, s), 4.84 (1H, s), 6.93-6.97 (2H, m), 7.13-7.19 (5H, m), 7.23-7.27 (4H, m). | 7.70; 468 |
| Anti (1S, 2S) 7-(2- Hydroxy-2,2-di- thiophen-2-yl- acetoxy)- bicyclo[2.2.1]hept-2- yl]- dimethyl-phenethyl- ammonium bromide | 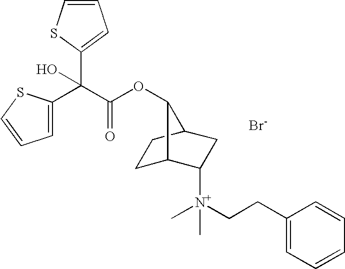 | (CDCl₃) 1.33-1.41 (1H, m), 1.46-1.68 (3H, m), 1.73-1.81 (1H, m), 2.12-2.23 (1H, m), 2.37-2.41 (1H, m), 2.77-2.81 (1H, m), 3.08-3.16 (2H, m), 3.30 (6H, s), 3.64-3.73 (2H, m), 4.40-4.48 (1H, m), 4.91 (1H, s), 5.04 (1H, s), 6.89- 6.94 (2H, m), 7.07-7.11 (2H, m), 7.15-7.26 (5H, m), 7.30- 7.34 (2H, m). | 7.84; 482 |
| Anti (1S, 2S) Hydroxy-diphenyl- acetic acid 2- (methyl-phenethyl- amino)-bicyclo[2.2.1] hept-7-yl ester | 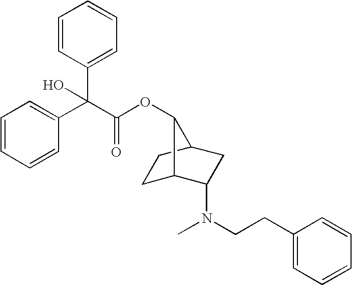 | (CDCl₃) 0.84-0.91 (1H, m), 1.04-1.15 (2H, m), 1.20-1.36 (1H, m), 1.65-1.80 (2H, m), 2.11-2.18 (4H, m), 2.29-2.34 (1H, m), 2.39-2.49 (3H, m), 2.62-2.75 (2H, m), 4.23 (1H, s), 4.85 (1H, s), 7.11-7.19 (3H, m), 7.21-7.34 (8H, m), 7.36-7.42 (4H, m). | 8.06; 456 |
| Anti (1S, 2S) [7-(2- Hydroxy-2,2- diphenyl-acetoxy)- bicyclo[2.2.1]hept-2- yl]-dimethyl- phenethyl- ammonium bromide | 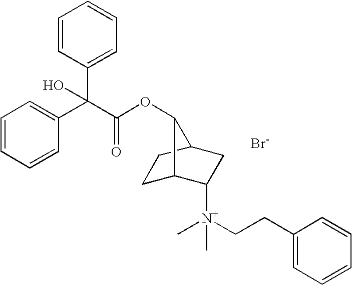 | (CDCl₃) 1.22-1.45 (3H, m), 1.62-1.71 (2H, m), 2.13-2.23 (1H, m), 2.38-2.41 (1H, m), 2.73-2.76 (1H, m), 3.08-3.15 (2H, m), 3.31 (3H, s), 3.34 (3H, s), 3.71-3.77 (2H, m), 4.17 (1H, s), 4.32-4.39 (1H, m), 4.91 (1H, s), 7.17-7.37 (15H, m). | 8.14; 470 |
| Anti (1S, 2S) Hydroxy-diphenyl- acetic acid 2- [methyl-(3-phenoxy- propyl)-amino]- bicyclo[2.2.1]hept- 7-yl ester | 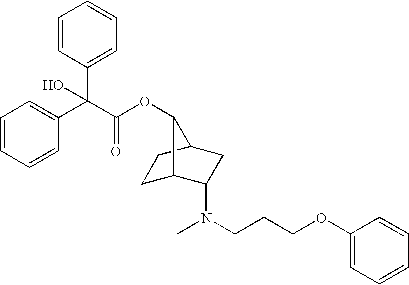 | (CDCl₃) 0.90-0.96 (1H, m), 1.01-1.11 (2H, m), 1.22-1.34 (1H, m), 1.67-1.80 (2H, m), 1.82-1.90 (2H, m), 2.04 (3H, s), 2.11-2.15 (1H, m), 2.26- 2.45 (4H, m), 3.90-4.01 (2H, m), 4.23 (1H, s), 4.85 (1H, s), 6.82-6.92 (3H, m), 7.21-7.33 (8H, m), 7.36-7.40 (4H, m). | 7.40; 486 |
| Anti (1S, 2S) [7-(2- Hydroxy-2,2- diphenyl-acetoxy)- bicyclo[2.2.1]hept-2- yl]-dimethyl- (3-phenoxy-propyl)- ammonium bromide | 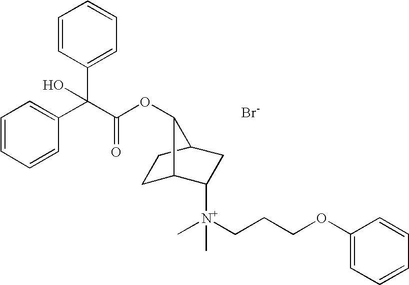 | (CDCl₃) 1.24-1.47 (3H, m), 1.60-1.73 (2H, m), 2.15-2.34 (3H, m), 2.39-2.43 (1H, m), 2.79-2.83 (1H, m), 3.28 (6H, s), 3.66-3.83 (2H, m), 4.02- 4.07 (2H, m), 4.22-4.31 (2H, m), 4.93 (1H, s), 6.79-6.83 (2H, m), 6.89-6.94 (1H, m), 7.19-7.38 (12H, m). | 8.50; 500 |
| Anti (1S, 2S) 9- Hydroxy-9H- xanthene-9- carboxylic acid 2- [methyl-(4-phenyl- butyl)-amino]- bicyclo[2.2.1]hept-7- yl ester | 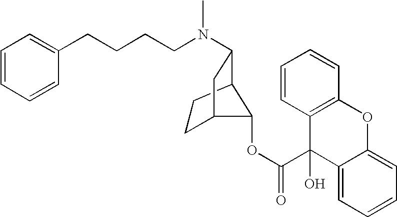 | (CDCl₃) 0.68-0.79 (1H, m), 0.80-0.87 (1H, m), 0.88-1.05 (2H, m), 1.35-1.45 (2H, m), 1.50-1.70 (4H, m), 1.90-1.98 (4H, m), 2.05-2.23 (4H, m), 2.55-2.60 (2H, m), 4.55 (1H, s), 4.90 (1H, s), 7.10-7.20(7H, m), 7.22-7.30 (2H, m), 7.32-7.39 (2H, m), 7.50-7.55 (2H, m). | 8.35; 498 |
| Anti (1S, 2S) [7-(9- Hydroxy-9H- xanthene-9- carbonyloxy)- bicyclo[2.2.1]hept-2- yl]-dimethyl-(4- phenyl-butyl)- ammonium bromide | 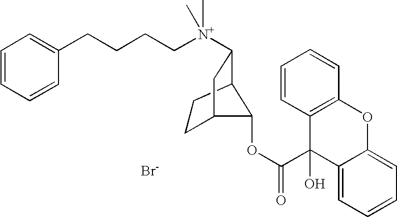 | (CDCl₃) 1.00-1.19 (3H, m), 1.42-1.56 (2H, m), 1.59-1.77 (4H, m), 1.96-2.10 (1H, m), 2.15-2.19 (1H, m), 2.40-2.43 (1H, m), 2.59-2.64 (2H, m), 3.13 (6H, s), 3.35-3.50 (2H, m), 4.17-4.22 (1H, m), 4.60 (1H, s), 5.00 (1H, s), 7.10- 7.28 (9H, m), 7.32-7.40 (2H, m), 7.51-7.59 (2H, m). | 8.01; 512 |
| Anti (1S, 2S) Hydroxy-di-thiophen- 2-yl-acetic acid 2-{[8-(tert- butoxycarbonyl- methyl-amino)- octyl]-methyl- amino}-bicyolo[2.2.1] hept-7-yl ester | 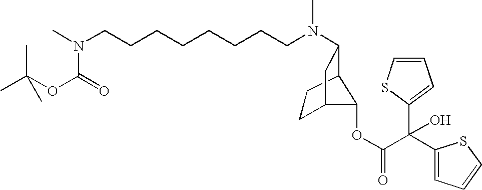 | (CDCl₃) 0.98-1.02 (1H, m), 1.20-1.60 (24H, m), 1.75-1.90 (2H, m), 2.05 (3H, s), 2.10- 2.30 (3H, m), 2.32-2.40 (2H, m), 2.85 (3H, s), 3.15-3.25 (2H, m), 2.78 (1H, s), 4.88 (1H, s), 6.95-7.0 (2H, m), 7.15-7.20 (2H, m), 7.25-7.30 (2H, m). | 9.02; 605 |
| Anti (1S, 2S) [8-(tert- Butoxycarbonyl- methyl-amino)- octyl]-[7-(2- hydroxy-2,2-di-thio phen-2-yl-acetoxy)- bicyclo[2.2.1]hept- 2-yl]-dimethyl- ammonium bromide | 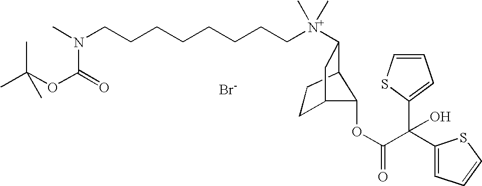 | (CDCl₃) 1.20-1.95 (26H, m), 2.20-2.30 (1H, m), 1.55 (1H, s), 2.40 (3H, s), 3.85 (1H, s), 3.15-3.20 (2H, m), 3.35 (3H, s), 3.37 (3H, s), 3.50-3.60 (2H, m), 4.30-4.40 (1H, m), 4.90 (1H, s), 4.95 (1H, s), 6.95-7.00 (2H, m), 7.10-7.15 (2H, m), 7.28-7.30 (2H, m). | 8.86; 619 |
| Anti (1S, 2S) [7-(2- Hydroxy-2,2-di- thiophen-2-yl- acetoxy)bicyclo[2.2.1] hept-2-yl]-dimethyl- (8-methylamino- octyl)-ammonium bromide | 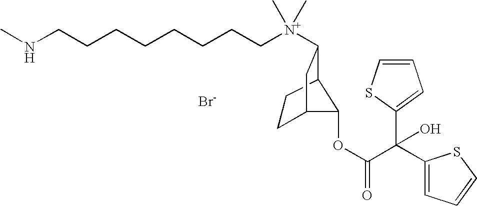 | — | 1.99; 519 (method 1) |

### Biological Examples

The inhibitory effects of compounds of the present invention at the M3 muscarinic receptor were determined by the following binding assays:

### Muscarinic Receptor Radioligand Binding Assays

Radioligand binding studies utilising [³H]-N-methyl scopolamine ([³H]-NMS) and commercially available cell membranes expressing the human muscarinic receptors (M2 and M3) were used to assess the affinity of muscarinic antagonists for M2 and M3 receptors. Membranes in TRIS buffer were incubated in 96-well plates with [³H]-NMS and M3 antagonist at various concentrations for 3 hours. Membranes and bound radioligand were then harvested by filtration and allowed to dry overnight. Scintillation fluid was then added and the bound radioligand counted using a Can berra Packard Topcount scintillation counter

The half-life of antagonists at each muscarinic receptor was measured using the alternative radioligand [³H]-QNB and an adaptation of the above affinity assay. Antagonists were incubated for 3 hours at a concentration 10-fold higher than their Ki, as determined with the [³H]-QNB ligand, with membranes expressing the human muscarinic receptors. At the end of this time, [³H]-QNB was added to a concentration 25-fold higher than its Kd for the receptor being studied and the incubation continued for various time periods from 15 minutes up to 180 minutes. Membranes and bound radioligand were then harvested by filtration and allowed to dry overnight. Scintillation fluid was then added and the bound radioligand counted using a Can berra Packard Topcount scintillation counter.

The rate at which [3H]-QNB is detected binding to the muscarinic receptors is related to the rate at which the antagonist dissociates from the receptor, ie. to the half life of the antagonists on the receptors.

All Example compounds that were tested in this assay showed binding affinity Kᵢ values of <10 nM, except for Example 6 which exhibited a Kᵢ value of <100 nM.

### Analysis of Inhibition of M3 Receptor Activation Via Calcium Mobilization

IN an alternative M3 receptor binding assay, CHO cells expressing the human M3 receptor were seeded and incubated overnight in 96 well collagen coated plates (black-wall, clear bottom) at a density of 50000/75 μL of medium in 3% serum. The following day, a calcium-sensitive dye (Molecular Devices, Cat # R8041) was prepared in HBSS buffer with the addition of 5 mM probenecid (pH 7.4). An equal volume of the dye solution (75 μL) was added to the cells and incubated for 45 minutes followed by addition of 50 μL of muscarinic antagonists or vehicle. After a further 15 minutes the plate was read on a FLEXstation™ (excitation 488 nm, emission 525 nm) for 15 seconds to determine baseline fluorescence. The muscarinic agonist Carbachol was then added at an EC₈₀ concentration and the fluorescence measured for a further 60 seconds. The signal was calculated by subtracting the peak response from the mean of the baseline fluorescence in control wells in the absence of antagonist. The percentage of the maximum response in the presence of antagonist was then calculated in order to generate IC₅₀ curves.

The inhibitory effects of compounds of the present invention at the M3 muscarinic Receptor may be evaluated in the following ex-viva and in vivo assays:

By way of illustration, the compound of Example 3 exhibited an IC50 value of <10 nM in this assay.

### Methacholine Induced Bronchoconstriction In Vivo

Male Guinea pigs (Dunkin Hartley), weighing 500-600 g housed in groups of 5 were individually identified. Animals were allowed to acclimatize to their local surroundings for at least 5 days. Throughout this time and study time animals were allowed access to water and food ad libitum.

Guinea pigs were anaesthetized with the inhaled anaesthetic Halothane (5%). Test compound or vehicle (0.25-0.50 mL/kg) was administered intranasally. Animals were placed on a heated pad and allowed to recover before being returned to their home cages.

Up to 24 hrs post dosing guinea pigs were terminally anaesthetized with Urethane (250 μg/mL, 2 mL/kg). At the point of surgical anaesthesia, the jugular vein was cannulated with a portex i.v. cannula filled with heparinised phosphate buffered saline (hPBS) (10 U/mL) for i.v. administration of methacholine. The trachea was exposed and cannulated with a rigid portex cannula and the oesophagus cannulated transorally with a flexible portex infant feeding tube.

The spontaneously breathing animal was then connected to a pulmonary measurement system (EMMS, Hants, UK) consisting of a flow pneumotach and a pressure transducer. The tracheal cannula was attached to a pneumotach and the oesophageal cannula attached to a pressure transducer.

The oesophageal cannula was positioned to give a baseline resistance of between 0.1 and 0.2 cmH20/mL/s. A 2 minute baseline reading was recorded before i.v. administration of methacholine (up to 30 μg/kg, 0.5 mL/kg). A 2 minute recording of the induced constriction was taken from the point of i.v. administration.

The software calculated a peak resistance and a resistance area under the curve (AUC) during each 2 minute recording period which was used to analyse the bronchoprotective effects of test compounds.

As an illustrative example, the compound of Example 3 was shown to be bronchoprotective 1 hr after dosing, shown in the accompanying drawings.

### BRIEF DESCRIPTION OF DRAWINGS

The accompanying drawing is a bar chart showing the effect of Example 3 (3 μg/kg, i.n.) on methacholine-induced bronchoconstriction in the Guinea pig.

1. A compound of formula (I): 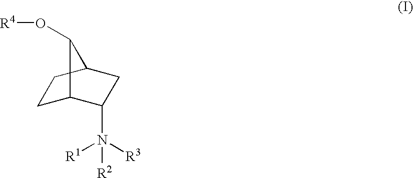
wherein
R¹ is C₁-C₆-alkyl or a hydrogen atom; and R² is a hydrogen atom or a group —R⁵, or a group, -Z-Y—R⁵, or a group -Z-NR⁹R¹⁰, or a group -Z-N(R⁹)C(O)R¹¹; and R³ is a lone pair, or C₁-C₆-alkyl in which case the nitrogen atom to which it is attached is a quaternary nitrogen and carries a positive charge;
R⁴ is selected from one of the groups of formula (a), (b), (c) or (d); 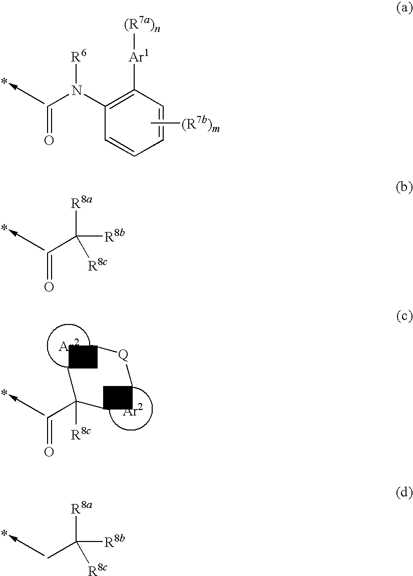
Z is a C₁-C₁₆-alkylene, C₂-C₁₆-alkenylene or C₂-C₁₆-alkynylene group;
Y is a bond or oxygen atom;
R⁵ is an C₁-C₆-alkyl, aryl, arylalkyl; aryl-fused-cycloalkyl, aryl-fused-heterocycloalkyl, heteroaryl, aryl(C₁-C₈-alkyl)-, heteroaryl(C₁-C₈-alkyl)-, cycloalkyl or heterocycloalkyl group;
R⁶ is C₁-C₆-alkyl or a hydrogen atom;
R^{7a} and R^{7b} are independently a C₁-C₆-alkyl group or halogen;
n and m are independently 0, 1, 2 or 3;
R^{8a} and R^{8b} are independently selected from the group consisting of aryl, aryl-fused-heterocycloalkyl, heteroaryl, C₁-C₆-alkyl, cycloalkyl and hydrogen;
R^{8c} is —OH, C₁-C₆-alkyl, hydroxy-C₁-C₆-alkyl, or a hydrogen atom;
R⁹ and R¹⁰ are independently a hydrogen atom, C₁-C₆-alkyl, aryl, aryl-fused-heterocycloalkyl, aryl-fused-cycloalkyl, heteroaryl, aryl(C₁-C₆-alkyl)-, or heteroaryl(C₁-C₆-alkyl)-group; or R⁹ and R¹⁰ together with the nitrogen atom to which they are attached form a heterocyclic ring of 4-8 atoms, optionally containing a further nitrogen or oxygen atom;
R¹¹ is C₁-C₆-alkyl or a hydrogen atom;
Ar¹ is aryl, heteroaryl or cycloalkyl;
Ar² are independently aryl, heteroaryl or cycloalkyl; and
Q is an oxygen atom, —CH₂—, —CH₂CH₂— or a bond;
or a pharmaceutically acceptable salt, solvate, N-oxide or prodrug thereof.

2. A compound as claimed in claim 1 wherein
R¹ is C₁-C₆-alkyl or a hydrogen atom and R² is C₁-C₆-alkyl, a hydrogen atom or a group -Z-Y—R⁵, or a group -Z-NR⁹R¹⁰
R³ is a lone pair; or C₁-C₆-alkyl, in which case the nitrogen atom to which it is attached is a quaternary nitrogen and carries a positive charge;
R⁴ is selected from one of the groups of formula (a) or (b) or (c): 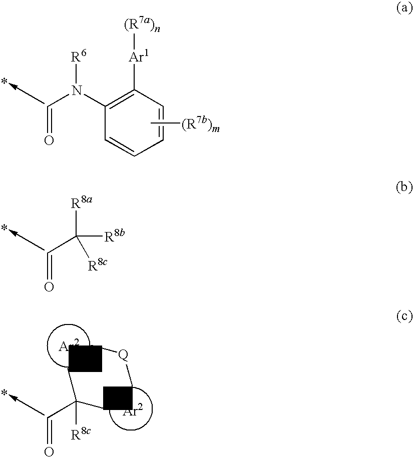
Z is a C₁-C₈-alkylene group;
Y is a bond or oxygen atom;
R⁵ is an aryl or aryl(C₁-C₈-alkyl)-group;
R⁶ is C₁-C₆-alkyl or a hydrogen atom;
R^{7a} and R^{7b} are independently a C₁-C₆-alkyl group or halogen;
n and m are independently 0, 1, 2 or 3;
R^{8a} and R^{8b} are independently selected from the group consisting of aryl, heteroaryl, C₁-C₆-alkyl, cycloalkyl and hydrogen;
R^{8c} is —OH, C₁-C₆-alkyl, hydroxy-C₁-C₆-alkyl, or a hydrogen atom;
R⁹ and R¹⁰ are independently a hydrogen atom, C₁-C₆-alkyl, aryl, heteroaryl, aryl(C₁-C₆-alkyl)-, or heteroaryl(C₁-C₆-alkyl)-group; or R⁹ and R¹⁰ together with the nitrogen atom to which they are attached form a heterocyclic ring of 4-8 atoms, optionally containing a further nitrogen or oxygen atom.

3. A compound as claimed in claim 1 wherein R¹ is methyl or ethyl, or a hydrogen atom and R² is methyl or ethyl, a hydrogen atom or a group -Z-Y—R⁵, or a group -Z-NR⁹R¹⁰.

4. A compound as claimed in claim 3 wherein R³ is methyl, so that the nitrogen atom to which it is attached is a quaternary nitrogen and carries a positive charge.

5. A compound as claimed in claim 1 wherein, in any group —R⁵, —Y—R⁵, -Z-Y—R⁵, -Z-NR⁹R¹⁰:
Z is —(CH₂)₁₋₁₆—, optionally substituted on up to three carbons in the chain by methyl;
Y is a bond or —O—;
R⁵ is optionally substituted: phenyl, 3,4-methylenedioxyphenyl, 3,4-ethylenedioxyphenyl, dihydrobenzofuranyl, naphthyl; or
pyridyl, pyrrolyl, pyrimidinyl, oxazolyl, isoxazolyl, benzisoxazolyl, benzoxazolyl, thiazolyl, benzthiazolyl, quinolyl, thienyl, benzthienyl, furyl, benzfuryl, imidazolyl, benzimidazolyl, isothiazolyl, benzisothiazolyl, pyrazolyl, isothiazolyl, triazolyl, benztriazolyl, thiadiazolyl, oxadiazolyl, pyridazinyl, pyridazinyl, triazinyl, indolyl or indazolyl; or
arylalkyl wherein the aryl part is phenyl, 3,4-methylenedioxyphenyl, 3,4-ethylenedioxyphenyl, dihydrobenzofuranyl, or naphthyl, and the alkyl part is —CH₂— or —CH₂CH₂—; or
heteroarylalkyl wherein the heteroaryl part is pyridyl, pyrrolyl, pyrimidinyl, oxazolyl, isoxazolyl, benzisoxazolyl, benzoxazolyl, thiazolyl, benzthiazolyl, quinolyl, thienyl, benzothienyl, furyl, benzofuryl, imidazolyl, benzimidazolyl, isothiazolyl, benzisothiazolyl, pyrazolyl, isothiazolyl, triazolyl, benztriazolyl, thiadiazolyl, oxadiazolyl, pyridazinyl, pyridazinyl, triazinyl, indolyl or indazolyl, and the alkyl part is —CH₂— or —CH₂CH₂—; or
indanyl or 1,2,3,4-tetrahydronaphthalenyl; or
heterocycloalkyl(C₁-C₆-alkyl)-, wherein the heterocycloalkyl part is azetidinyl, piperidinyl, piperazinyl, N-substituted piperazinyl such as methylpiperazinyl, or tetrahydropyrrolyl and the alkyl part is —CH₂— or —CH₂CH₂—. or
cyclopropyl, cyclobutyl, cyclopentyl, cyclohexyl, cycloheptyl or cyclooctyl; and
R⁹ and R¹⁰ are independently
hydrogen; methyl, ethyl or n- or isopropyl;
phenyl, 3,4-methylenedioxyphenyl, 3,4-ethylenedioxyphenyl, dihydrobenzofuranyl, naphthyl;
pyridyl, pyrrolyl, pyrimidinyl, oxazolyl, isoxazolyl, benzisoxazolyl, benzoxazolyl, thiazolyl, benzthiazolyl, quinolyl, thienyl, benzthienyl, furyl, benzfuryl, imidazolyl, benzimidazolyl, isothiazolyl, benzisothiazolyl, pyrazolyl, isothiazolyl, triazolyl, benztriazolyl, thiadiazolyl, oxadiazolyl, pyridazinyl, pyridazinyl, triazinyl, indolyl or indazolyl; or
arylalkyl wherein the aryl part is phenyl, 3,4-methylenedioxyphenyl, 3,4-ethylenedioxyphenyl, dihydrobenzofuranyl, or naphthyl, and the alkyl part is —CH₂— or —CH₂CH₂—; or R⁹ and R¹⁰ together with the nitrogen atom to which they are attached form an azetidinyl, piperidinyl, piperazinyl, N-methylpiperazinyl, pyrrolidinyl, morpholinyl, or thiomorpholinyl ring.

6. A compound as claimed in claim 1 wherein, in the group —NR¹R²R³, R¹ is methyl or ethyl, R² is a group -Z-Y—R⁵, and R³ is methyl, so that the nitrogen to which it is attached is quaternised and carries a positive charge.

7. A compound as claimed in claim 6 wherein R⁵ is optionally substituted phenyl, Y is a bond or —O—, and -Z- is a straight or branched alkylene radical linking the nitrogen and —YR⁵ by a chain of up to 12, or up to 9, carbon atoms.

8. A compound as claimed in claim 1 wherein R⁴ is a group (a), R⁶ is methyl or ethyl or a hydrogen atom; Ar¹ is phenyl, thienyl, cyclohexyl, cyclopentyl, cyclopropyl, or cyclobutyl; R^{7a} and R^{7b} are independently methyl, ethyl, n- or isopropyl, n-, sec- or tertbutyl, fluoro, chloro or bromo; and m and n are independently 0, 1, 2 or 3.

9. A compound as claimed in claim 1 wherein R⁴ is a group (b) and R^{8a} and R^{8b} may be independently selected from methyl, ethyl, n- or isopropyl, n-, sec- and tertbutyl; phenyl, 3,4-methylenedioxyphenyl, 3,4-ethylenedioxyphenyl, dihydrobenzofuranyl, naphthyl; pyridyl, pyrrolyl, pyrimidinyl, oxazolyl, isoxazolyl, benzisoxazolyl, benzoxazolyl, thiazolyl, benzthiazolyl, quinolyl, thienyl, benzthienyl, furyl, benzfuryl, imidazolyl, benzimidazolyl, isothiazolyl, benzisothiazolyl, pyrazolyl, isothiazolyl, triazolyl, benztriazolyl, thiadiazolyl, oxadiazolyl, pyridazinyl, pyridazinyl, triazinyl, indolyl or indazolyl; indanyl and 1,2,3,4-tetrahydronaphthalenyl; cyclopropyl, cyclobutyl, cyclopentyl, cyclohexyl, cycloheptyl and cyclooctyl and a hydrogen atom; R^{8c} is —OH, a hydrogen atom, methyl, ethyl, or hydroxymethyl.

10. A compound as claimed in claim 9 wherein R^{8c} is —OH.

11. A compound as claimed in claim 9 wherein (i) each of R^{8a} and R^{8b} is optionally substituted pyridyl, oxazolyl, thiazolyl, furyl, thienyl or phenyl; or (ii) one of R^{8a} and R^{8b} is optionally substituted phenyl and the other is cyclopropyl, cyclobutyl, cyclopentyl, cyclohexyl, cycloheptyl or cyclooctyl; or (iii) one of R^{8a} and R^{8b} is optionally substituted pyridyl, thienyl, oxazolyl, thiazolyl, or furyl and the other is cyclopropyl, cyclobutyl, cyclopentyl, cyclohexyl, cycloheptyl and cyclooctyl.

12. A compound as claimed in claim 9 wherein each of R^{8a} and R^{8b} is 2-thienyl or phenyl; or one of R^{8a} and R^{8b} is phenyl and the other is 2-thienyl.

13. A compound as claimed in claim 1 having the formula (IA) 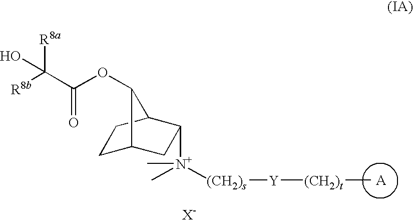
wherein ring A is an optionally substituted phenyl ring; R^{8a} is phenyl, thienyl, cyclopentyl or cyclohexyl; R^{8b} is phenyl; thienyl, cyclopentyl or cyclohexyl; s is 1, 2, 3, 4, 5, 6 or 7 and t is 0, 1, 2, 3, 4, 5, 6 or 7, provided that s+t is not greater than 10; Y is a bond or —O—, and X⁻ is a pharmaceutically acceptable anion.

14. A compound as claimed in claim 1 having the formula (IB) 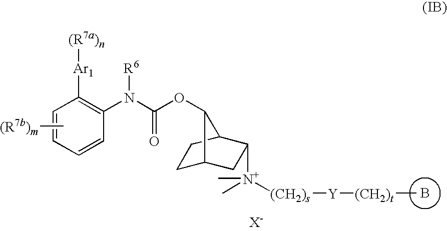
wherein ring B is an optionally substituted phenyl ring; s is 1, 2, 3, 4, 5, 6 or 7 and t is 0, 1, 2, 3, 4, 5, 6 or 7 provided that s+t is not greater than 10; Y is a bond or —O—; and R⁶, Ar¹, R and R^{7b} are as defined for group (a) above; and X⁻ is a pharmaceutically acceptable anion.

15. A compound as claimed in claim 1 having the formula (IC) 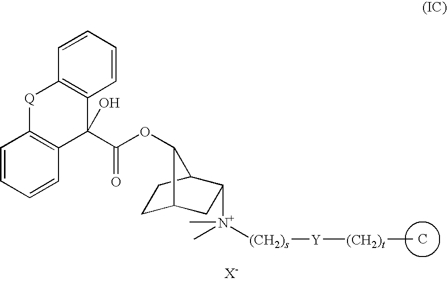
wherein ring C is an optionally substituted phenyl ring; Q is an oxygen atom, —CH₂—, —CH₂CH₂— or a bond; s is 1, 2, 3, 4, 5, 6 or 7 and t is 0, 1, 2, 3, 4, 5, 6 or 7 provided that s+t is not greater than 10 and Y is a bond or —O—; and X⁻ is a pharmaceutically acceptable anion.

16. A compound as claimed in claim 13 wherein optional substituents in the ring A or B or C as the case may be selected from alkoxy, halo especially fluoro or chloro, C₁-C₃-alkyl, amino C₁-C₃-acyl, amino C₁-C₃-alkyl, and aminosulfonyl.

17. A compound as claimed in claim 1 which is, or is predominantly, in the anti-endo configuration.

18. (canceled)

19. A pharmaceutical composition comprising a compound as claimed in claim 1 and a pharmaceutically acceptable carrier or excipient.

20. A pharmaceutical composition as claimed in claim 19 in a form suitable for inhalation.

21. (canceled)

22. A method of treatment of a disease or condition in which M3 muscarinic receptor activity is implicated comprising administration to a subject in need thereof of an effective amount of a compound as claimed in claim 1.

23. The method of treatment as claimed in claim 22, wherein the disease or condition is a respiratory-tract disorder.

24. The method of treatment as claimed in claim 22, wherein the disease or condition is a gastrointestinal-tract disorder.

25. The method of treatment as claimed in claim 22, wherein the disease or condition is a cardiovascular disorder.

26. The method of treatment as claimed in claim 22, wherein the disease or condition is chronic obstructive lung disease, chronic bronchitis, asthma, chronic respiratory obstruction, bronchial hyperactivity, pulmonary fibrosis, pulmonary emphysema, or allergic rhinitis.

27. The method of treatment as claimed in claim 22, wherein the disease or condition is irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakiuria, neurogenic bladder, nocturnal enuresis, psychosomatic bladder, incontinence associated with bladder spasms or chronic cystitis, urinary urgency or pollakiuria; motion sickness; and cardiovascular disorders such as vagally induced sinus bradycardia.

28. The method of treatment as claimed in claim 22, wherein the disease or condition is vagally induced sinus bradycardia.
